(19) United States
(12) Patent Application Publication
(10) Pub. No.: US 2018/0291377 A1
(43) Pub. Date: Oct. 11, 2018

(54) Blood Brain Barrier Opening Agents and Uses Thereof

(71) Applicants: Jianghui Hou, St. Louis, MO (US); Yong-feng Gong, St. Louis, MO (US)

(72) Inventors: Jianghui Hou, St. Louis, MO (US); Yong-feng Gong, St. Louis, MO (US)

(73) Assignee: Washington University, St. Louis, MO (US)

(21) Appl. No.: 15/950,392

(22) Filed: Apr. 11, 2018

Related U.S. Application Data
(60) Provisional application No. 62/484,071, filed on Apr. 11, 2017.

(51) Int. Cl. C12N 15/113; A61K 31/713; A61K 31/495; A61K 31/704

(52) U.S. Cl. CPC C12N 15/113; A61K 31/713; C12N 2310/14; A61K 31/704; A61K 31/495

(57) ABSTRACT

Among the various aspects of the present disclosure is the provision of a blood brain barrier (BBB) opening agent and uses thereof. An aspect of the present disclosure provides for methods of opening up the BBB; increasing the permeability of the tricellular junction; and using a BBB opening agent for the treatment of a brain pathology or neurological disease, disorder, or condition.

### CROSS-REFERENCE TO RELATED APPLICATIONS

This application claims priority from U.S. Provisional Application Ser. No. 62/484,071 filed on 11 Apr. 2017, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to methods and compositions for use in opening the blood brain barrier and drug delivery to the brain.

### BACKGROUND OF THE INVENTION

Brain tumors are known to be the most lethal disease known to mankind. Approximately 170,000 people in the USA are diagnosed with primary brain tumors every year; more than 13,000 of these patients die each year. Over half of a million new patients are diagnosed with metastasized secondary brain tumors in the USA. The number of patients diagnosed with metastasized brain tumors is 3-fold larger than the primary tumor patients. The average lifespan of patients diagnosed with brain tumors are less than 24 months, even after surgery.

Gliobastoma is the most common (accounting for 45-50% of all gliomas) and aggressive of all gliomas. Every year, there are approximately 20,000 new patients diagnosed with gliobastoma. Due to its infiltrative nature, glioblastomas are presented with very poor prognosis. More than 13,000 of these patients die each year. Median survival for patients offered only supportive care is approximately 14 weeks. Maximal surgical resection can only extend median survival to approximately 15-18 months.

A number of chemotherapy reagents have been approved including temozolomide (194 Da). However, due the presence of the blood brain barrier, even small molecule drugs such as temozolomide minimally reach the tumor cells and only extend the lifespan of patients by 3-4 months. Other more effective cancer drugs such as doxorubicin (with a molecule weight of 544 Da) were completely impermeable to the blood brain barrier, and thus unable to kill the tumor cells.

### SUMMARY OF THE INVENTION

Among the various aspects of the present disclosure is the provision of a composition comprising a blood brain barrier (BBB) opening agent and processes of making and using the same. Briefly, the present disclosure includes compositions and methods directed to opening the BBB to allow drugs to more effectively reach the brain parenchyma.

In one aspect, the present disclosure provides for methods for treating a brain pathology.

In yet another aspect, the present disclosure provides for methods for opening a blood brain barrier (BBB).

In yet another aspect, the present disclosure provides for compositions comprising a blood brain barrier agent.

In some embodiments, the methods can include administering a therapeutically effective amount of a blood brain barrier opening agent. For example, the therapeutically effective amount of the blood brain barrier opening agent can increase permeability of the tricellular junction.

In some embodiments, the methods can include a blood brain barrier opening agent comprising a synthetic RNA molecule, a RNA interference molecule, a siRNA, an antibody synthesized against angulin, or a small molecule inhibitor of angulin; an angulin inhibitor or an anti-angulin antibody; a synthetic RNA molecule, a RNA interference molecule, or a siRNA synthesized against angulin; or a monoclonal antibody, a polyclonal antibody, or an antigen binding fragment thereof comprising an antigen binding site that binds specifically to a LSR or ILDR polypeptide.

In some embodiments, the methods can include a blood brain barrier opening agent comprising an anti-LSR (lipolysis stimulated lipoprotein receptor) antibody or an anti-ILDR (immunoglobulin-like domain containing receptor), an antigen binding fragment thereof, or a functional equivalent thereof, or a nucleic acid encoding the antibody thereof; an RNAi molecule directed to LSR or ILDR, or a polynucleotide encoding the RNAi molecule; an anti-LSR antibody that specifically binds to an epitope of the LSR; an anti-ILDR antibody that specifically binds to an epitope of the ILDR; or an anti-LSR antibody or anti-ILDR antibody is a monoclonal antibody; an anti-LSR antibody or anti-ILDR antibody is an antibody selected from the group consisting of: a monoclonal antibody, polyclonal antibody, chimeric antibody, humanized antibody, human antibody, multifunctional antibody, bispecific or oligospecific antibody, single chain antibody, scFV, diabody, sc(Fv)2 (single chain (Fv)2), and scFv-Fc.

In some embodiments, the methods can include a blood brain barrier opening agent comprising an LSR siRNA or an ILDR siRNA, wherein the LSR siRNA reduces LSR cerebral expression; or the ILDR siRNA reduces ILDR cerebral expression.

In some embodiments, the methods can include a functional siRNA duplex molecule comprising sense and anti-sense strands selected from one or more of the group consisting of: SEQ ID NO: 1 or a sequence 90% identical thereto and SEQ ID NO: 2 or a sequence 90% identical thereto; SEQ ID NO: 3 or a sequence 90% identical thereto and SEQ ID NO: 4 or a sequence 90% identical thereto; SEQ ID NO: 5 or a sequence 90% identical thereto and SEQ ID NO: 6 or a sequence 90% identical thereto; and SEQ ID NO: 7 or a sequence 90% identical thereto and SEQ ID NO: 8 or a sequence 90% identical thereto; and the functional siRNA duplex molecule has siRNA activity against angulin.

In some embodiments, the methods can include a blood brain barrier opening agent comprising or coupled to a drug, a radionuclide, an enzyme, a toxin, a therapeutic agent, or a chemotherapeutic agent.

In some embodiments, the methods can include a pharmaceutically acceptable excipient, a preservative, a water solubility enhancing reagent, a label, or a tag.

In some embodiments, the methods can include administering a therapeutically effective amount of a therapeutic agent, wherein the therapeutic agent crosses the blood brain barrier in an increased amount compared to a control not receiving the BBB opening agent

In some embodiments, the methods can include a cancer treatment or a chemotherapeutic agent for the treatment of a brain tumor.

In some embodiments, the methods can include radiation therapy, antibody therapy, chemotherapy, photodynamic therapy, adoptive T cell therapy, T_{reg} depletion, surgery, or a combination therapy with conventional drugs.

In some embodiments, the methods can include a cytotoxic drug, a tumor vaccine, an antibody selected from the group consisting of bevacizumab, Erbitux® (cetuximab), and immunostimulatory antibodies; peptides, pepti-bodies, small molecules, a chemotherapeutic agent, interferons, interleukins, growth hormones, folic acid, vitamins, minerals, aromatase inhibitors, RNAi, histone deacetylase inhibitors, or proteasome inhibitors.

In some embodiments, the methods can include a cytotoxic agent or a cytostatic agent. In accordance with yet another aspect, the methods of treatment or opening of the BBB can include paclitaxel, cisplatin, vinorelbine, docetaxel, gemcitabine, temozolomide, irinotecan, 5FU, or carboplatin. In accordance with yet another aspect, the methods of treatment or opening of the BBB can include a subject diagnosed or suspected of having a brain cancer, a brain tumor, a spinal cord cancer, a spinal cord tumor, a neurodegenerative disease, multiple sclerosis, a stroke, Alzheimer's disease, Acoustic Neuroma; Astrocytoma (e.g., Grade I—Pilocytic Astrocytoma, Grade II—Low-grade Astrocytoma, Grade III—Anaplastic Astrocytoma, Grade IV—Glioblastoma (GBM), a juvenile pilocytic astrocytoma); Atypical Teratoid Rhaboid Tumor (ATRT); Chordoma; Chondrosarcoma; Choroid Plexus; CNS Lymphoma; Craniopharyngioma; cysts; Ependymoma; Ganglioglioma; Germ Cell Tumor, Glioblastoma (GBM); Gliomas (e.g., Brain Stem Glioma, Ependymoma, Mixed Glioma, Optic Nerve Glioma, Subependymoma); Hemangioma; Lipoma; Lymphoma; Medulloblastoma; Meningioma; Metastatic Brain Tumors; Neurofibroma; Neuronal & Mixed Neuronal-Glial Tumors; Non-Hodgkin lymphoma; Oligoastrocytoma; Oligodendroglioma; Pineal Tumors; Pituitary Tumors; Primitive Neuroectodermal (PNET); Other Brain-Related Conditions; Schwannoma (neurilemmomas); Brain Stem Glioma; Craniopharyngioma; Ependymoma; Juvenile Pilocytic Astrocytoma (JPA); Medulloblastoma; Optic Nerve Glioma; Pineal Tumor; Primitive Neuroectodermal Tumors (PNET); or Rhabdoid Tumor.

In some embodiments, the methods can include the slowed progression or amelioration of a brain pathology, a brain tumor, a brain cancer, a spinal cord tumor, a spinal cord cancer, or a neurological disease; or the blood brain barrier opening agent does not cause global tight junction breakdown in the brain or lead to neuro-inflammation

In some embodiments, the methods can include an increase in the level of a therapeutic agent in the brain of a subject administered with a BBB opening agent compared to a control subject not receiving a BBB opening agent.

In yet another aspect, the present disclosure provides for methods of producing a synthetic siRNA molecule against angulin.

In some embodiments, the methods of producing a synthetic RNA molecule against angulin can include providing a single stranded sense RNA molecule; providing a single stranded anti-sense RNA molecule; and combining the single stranded sense RNA molecule and the single stranded anti-sense RNA molecule, forming a functional siRNA duplex molecule; and the functional siRNA duplex molecule has siRNA activity against angulin.

In some embodiments, the methods of producing a synthetic RNA molecule against angulin can include a functional siRNA duplex molecule comprising sense and anti-sense strands selected from one or more of the group consisting of: SEQ ID NO: 1 or a sequence 90% identical thereto and SEQ ID NO: 2 or a sequence 90% identical thereto; SEQ ID NO: 3 or a sequence 90% identical thereto and SEQ ID NO: 4 or a sequence 90% identical thereto; SEQ ID NO: 5 or a sequence 90% identical thereto and SEQ ID NO: 6 or a sequence 90% identical thereto; or SEQ ID NO: 7 or a sequence 90% identical thereto and SEQ ID NO: 8 or a sequence 90% identical thereto.

In some embodiments, the methods of producing a synthetic RNA molecule against angulin can include single stranded sense and antisense RNA molecules are chemically synthesized by automated solid phase oligonucleotide synthesizer; or the combining step comprises approximately molar equivalents of the sense and anti-sense strands.

In some embodiments, the methods of producing a synthetic RNA molecule against angulin can include combining the functional siRNA duplex molecule with a liposome reagent, forming an in vivo-grade siRNA molecule.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a bar graph showing mRNA expression level in a control mouse (receiving a scrambled siRNA injection) and a mouse receiving LSR siRNA injection (KD) (see e.g., Example 2). LSR siRNA injection significantly reduced the LSR cerebral expression by 60% (p<0.05).

FIG. 2 is a series of immunofluorescence staining images labeled with anti-LSR antibody showing cerebral cortical sections from mice receiving siRNA#736+siRNA #2119 (KD) or scrambled siRNA (control) injections (see e.g., Example 3). Note the tricellular tight junction in the cerebral capillary blood vessels (arrow). Note that KD samples showed reduced LSR protein labeling intensity.

FIG. 3 is a bar graph showing fluorescein levels in the brain of a control mouse (receiving a scrambled siRNA injection) and a mouse receiving a LSR siRNA injection (KD) (siRNA #736+siRNA #2119) injected with fluorescein (see e.g., Example 4).

FIG. 4 is a bar graph showing temozolomide levels in mice receiving LSR siRNA injection (KD) (siRNA #736+siRNA #2119) and mice receiving a scrambled siRNA injection (Control) (see e.g., Example 5).

FIG. 5 is a bar graph showing doxorubicin levels in mice receiving LSR siRNA injection (KD) (siRNA #736+siRNA #2119); N=4 mice receiving scrambled siRNA injection (Control) (see e.g., Example 6).

FIG. 6 is a bar graph showing mRNA expression level for cell transfections receiving LSR siRNAs (KD) (siRNA #216+siRNA #822); N=4 cell transfections receiving scrambled siRNA (Control) (see e.g., Example 7).

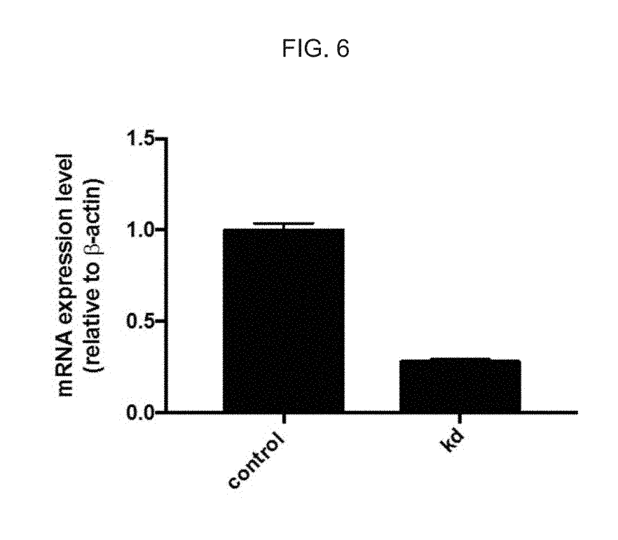

FIG. 7 is a bar graph showing fluorescein levels for N=4 cell transfections receiving LSR siRNAs (KD) (siRNA #216+siRNA #822); N=4 cell transfections receiving scrambled siRNA (Control) (see e.g., Example 8).

FIG. 8 is a bar graph showing Temozolomide levels for N=4 cell transfections receiving LSR siRNAs (KD) (siRNA #216+siRNA #822); N=4 cell transfections receiving scrambled siRNA (Control) (see e.g., Example 9).

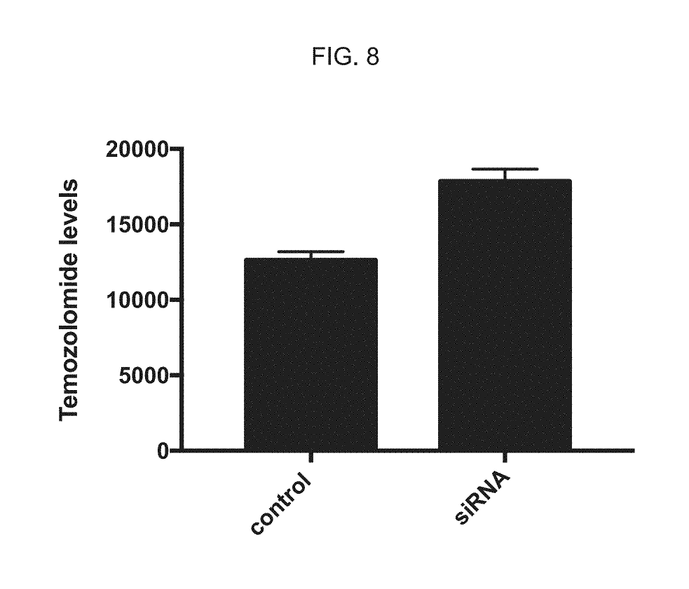

FIG. 9 is a bar graph showing doxorubicin levels for N=4 cell transfections receiving LSR siRNAs (KD) (siRNA #216+siRNA #822); N=4 cell transfections receiving scrambled siRNA (Control) (see e.g., Example 10).

### DETAILED DESCRIPTION OF THE INVENTION

The present disclosure is based, at least in part, on the discovery that administration of siRNA against angulin (e.g., LSR) opens up the tricellular junction and increases permeability to the brain.

As described herein, manipulating tricellular junction permeability in the blood brain barrier can facilitate treatment of brain pathologies, e.g., brain tumors. As shown herein, the study of the tight junction biology of the blood brain barrier has established the following: (1) the tricellular tight junction has different permeability profiles compared to the bicellular tight junction; (2) the tricellular tight junction is responsible for large size organic molecule permeation while the bicellular tight junction is for small size inorganic molecules such as ions; (3) deletion of the angulin protein from tricellular tight junction increases permeability of large molecules such as cancer drugs, temozolomide (194 Da) and doxorubicin (544 Da); (4) deletion of angulin protein did not affect the bicellular tight junction function or the overall TJ barrier structure; and (5) deletion of the angulin protein increases the temozolomide and doxorubicin permeability to cerebral cortex in live mice in vivo.

The present disclosure provides for the identification of effective siRNA molecules; IV injection of siRNA molecules in mice demonstrates that it can open the blood brain barrier after injecting siRNA the mouse brain, data shows the BBB is significantly more permeable to temozolomide and doxorubicin. The present disclosure further provides in vivo (mouse) and in vitro (human cell) data that demonstrates angulin targeted siRNA molecules reduce mRNA expression; reduce angulin protein expression; and increase permeability of the BBB to allow molecules such as fluorescein (FITC), temozolomide, and doxorubicin.

Brain Pathology

The methods and compositions as described herein can increase drug delivery to the brain. For example, the drug to be delivered to the brain can be a drug suitable for treating a brain pathology. For example, the methods and compositions as described herein can improve known methods of treatment for a brain pathology by allowing a drug or a therapeutic agent to reach the brain parenchyma by opening up the blood brain barrier. A brain pathology that can be treated with the disclosed compositions and methods can be a disease, disorder, or condition of the brain, such as brain cancer, a brain tumor, or any other neurological disorder, disease, or condition.

Brain and Spinal Cord Tumors or Cancer

The present disclosure provides for methods and compositions for use in the treatment of brain tumors, brain cancer, or spinal cord tumors. For example, a brain or spinal cord tumor that can be treated with the methods and compositions as described herein can be Acoustic Neuroma; Astrocytoma (e.g., Grade I—Pilocytic Astrocytoma, Grade II—Low-grade Astrocytoma, Grade III—Anaplastic Astrocytoma, Grade IV—Glioblastoma (GBM), a juvenile pilocytic astrocytoma); Atypical Teratoid Rhaboid Tumor (ATRT); Chordoma; Chondrosarcoma; Choroid Plexus; CNS Lymphoma; Craniopharyngioma; cysts; Ependymoma; Ganglioglioma; Germ Cell Tumor; Glioblastoma (GBM); Gliomas (e.g., Brain Stem Glioma, Ependymoma, Mixed Glioma, Optic Nerve Glioma, Subependymoma); Hemangioma; Lipoma; Lymphoma; Medulloblastoma; Meningioma; Metastatic Brain Tumors; Neurofibroma; Neuronal & Mixed Neuronal-Glial Tumors; Non-Hodgkin lymphoma; Oligoastrocytoma; Oligodendroglioma; Pineal Tumors; Pituitary Tumors; Primitive Neuroectodermal (PNET); Other Brain-Related Conditions; Schwannoma (neurilemmomas); Brain Stem Glioma; Craniopharyngioma; Ependymoma; Juvenile Pilocytic Astrocytoma (JPA); Medulloblastoma; Optic Nerve Glioma; Pineal Tumor, Primitive Neuroectodermal Tumors (PNET); or Rhabdoid Tumor.

Neurological Diseases. Disorders, or Conditions

The present disclosure provides for methods and compositions for use in the treatment of neurological diseases, disorders, or conditions. For example, a neurological disease, disorder, or condition that can be treated with the methods and compositions as described herein can be Abulia; Agraphia; Alcoholism; Alexia; Alien hand syndrome; Allan-Hemdon-Dudley syndrome; Alternating hemiplegia of childhood; Alzheimer's disease; Amaurosis fugax; Amnesia; Amyotrophic lateral sclerosis (ALS); Aneurysm; Angelman syndrome; Anosognosia; Aphasia; Apraxia; Arachnoiditis; Amold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder; Bell's palsy; Brachial plexus injury; Brain damage; Brain injury; Brain tumor; Brody myopathy; Canavan disease; Capgras delusion; Carpal tunnel syndrome: Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation; Chorea; Chronic fatigue syndrome: Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cyclothymic disorder; Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis; Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting; Familial spastic paralysis; Febrile seizures; Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis; Giant cell inclusion disease; Globoid Cell Leukodystrophy; Gray matter heterotopia; Guillain-Barre syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster Hirayama syndrome; Hirschsprung's disease; Holmes-Adie syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Isodicentric 15; Joubert syndrome; Karak syndrome; Kearns-Sayre syndrome; Kinsboume syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease (e.g., amyotrophic lateral sclerosis); Lumbar disc disease; Lumbar spinal stenosis; Lyme disease—Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy; Morvan syndrome; Motor Neurone Disease (e.g., amyotrophic lateral sclerosis); Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neuro-Behçet's disease; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myodonus syndrome; Optic neuritis; Orthostatic Hypotension; Otosclerosis; Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy; Ramsay Hunt syndrome type I; Ramsay Hunt syndrome type II; Ramsay Hunt syndrome type III (e.g., Ramsay-Hunt syndrome); Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schilder's disease (two distinct conditions); Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury; Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome.

Blood Brain Barrier Opening Agent

The present disclosure provides for compositions comprising and uses of a blood brain barrier opening agent (BBB opening agent).

For example, the BBB opening agent can comprise an RNA interference molecule, siRNA, an antibody, or a small molecule inhibitor. As an example, the blood brain barrier opening agent can comprise a siRNA against angulin, an angulin inhibitor, or an angulin antibody.

As another example, the present disclosure provides for a method to regulate the tTJ permeability by combining BBB opening agents, such as siRNAs (e.g., against the LSR (angulin-1) gene) with an anti-cancer therapeutic (e.g., temozolomide or doxorubicin) into intravenous injections and shows that such a method can significantly increase the permeability of an anti-cancer therapeutic (e.g., temozolomide or doxorubicin) across the BBB into the brain parenchyma. The present disclosure further presents a new route, via a BBB opening agent, to deliver important cancer drugs into brain parenchyma to treat brain tumors that are normally unreachable due the blood brain barrier.

As described herein the BBB opening agent increases the amount of a therapeutic agent into the brain of a subject by about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; about 100%; about 101%; about 102%; about 103%; about 104%; about 105%; about 106%; about 107%; about 108%; about 109%; about 110%; about 111%; about 112%; about 113%; about 114%; about 115%; about 116%; about 117%; about 118%; about 119%; about 120%; about 121%; about 122%; about 123%; about 124%; about 125%; about 126%; about 127%; about 128%; about 129%; about 130%; about 131%; about 132%; about 133%; about 134%; about 135%; about 136%; about 137%; about 138%; about 139%; about 140%; about 141%; about 142%; about 143%; about 144%; about 145%; about 146%; about 147%; about 148%; about 149%; about 150%; about 151%; about 152%; about 153%; about 154%; about 155%; about 156%; about 157%; about 158%; about 159%; about 160%; about 161%; about 162%; about 163%; about 164%; about 165%; about 166%; about 167%; about 168%; about 169%; about 170%; about 171%; about 172%; about 173%; about 174%; about 175%; about 176%; about 177%; about 178%; about 179%; about 180%; about 181%; about 182%; about 183%; about 184%; about 185%; about 186%; about 187%; about 188%; about 189%; about 190%; about 191%; about 192%; about 193%; about 194%; about 195%; about 196%; about 197%; about 198%; about 199%; or about 200% or more than about 100%, more than about 200%, more than about 300%, or more than about 400% when compared to a subject that was not given the BBB opening agent. Recitation of each of these discrete values is understood to include ranges between each value.

As described herein the BBB opening agent can reduce angulin expression in the brain by about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100% when compared to a subject that was not given the BBB opening agent. Recitation of each of these discrete values is understood to include ranges between each value.

As described herein the BBB opening agent can reduce mRNA expression level (relative to β-actin) by about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100% when compared to a subject that was not given the BBB opening agent. Recitation of each of these discrete values is understood to include ranges between each value.

Angulin

Angulin is a family of Tricellular TJ family (3 members) (see e.g., Sohet et al., J. Cell Biol. 208(6):703-711 (2015)). There are 3 isoforms of the angulin gene (angulin-1, angulin-2, and angulin-3).

Angulin-1 is also known as Lipolysis-stimulated lipoprotein receptor (LSR). Aliases for LSR Gene (Q86X29; see e.g., LSR GeneCard) can be Lipolysis Stimulated Lipoprotein Receptor; Immunoglobulin-Like Domain Containing Receptor 3; Lipolysis-Stimulated Remnant; Liver-Specific BHLH-Zip Transcription Factor; Lipolysis-Stimulated Lipoprotein Receptor; LISCH Protein, LISCH7; ILDR3; or LISCH.

Angulin-2 is also known as Immunoglobulin-like domain containing receptor-1 (ILDR1).

Angulin-3 is also known as Immunoglobulin-like domain containing receptor-2 (ILDR2).

LSR has 6 isoforms produced by alternative splicing.

```
LSR Isoform 1 (identifier Q86X29-1)
(SEQ ID NO: 9)
                10 20 30 40 50
MQQDGLGVGT RNGSGKGRSV HPSWPWCAPR PLRYFGRDAR ARRAQTAAMA
                60 70 80 90 100
LLAGGLSRGL GSHPAAAGRD AVVFVWLLLS TWCTAPARAI QVTVSNPYHV
              110 120 130 140 150
VILFQPVTLP CTYQMTSTPT QPIVIWKYKS FCRDRIADAF SPASVDNQLN
              160 170 180 190 200
AQLAAGNPGY NPYVECQDSV RTVRVVATKQ GNAVTLGDYY QGRRITITGN
              210 220 230 240 250
ADLTFDQTAW GDSGVYYCSV VSAQDLQGNN EAYAELIVLG RTSGVAELLP
              260 270 280 290 300
GFQAGPIEDW LFVVVVCLAA FLIFLLLGIC WCQCCPHTCC CYVRCPCCPD
              310 320 330 340 350
KCCCPEALYA AGKAATSGVP SIYAPSTYAH LSPAKTPPPP AMIPMGPAYN
              360 370 380 390 400
GYPGGYPGDV DRSSSAGGQG SYVPLLRDTD SSVASEVRSG YRIQASQQDD
              410 420 430 440 450
SMRVLYYMEK ELANFDPSRP GPPSGRVERA MSEVTSLHED DWRSRPSPGP
              460 470 480 490 500
ALTPIRDEEW GGHSPRSPRG WDQEPAREQA GGGWRARRPR ARSVDALDDL
              510 520 530 540 550
TPPSTAESGS RSPTSNGGRS RAYMPPRSRS RDDLYDQDDS RDFPRSRDPH
              560 570 580 590 600
YDDFRSRERP RADPRSHHHR TRDPRDNGSR SGDLPYDGRL LEEAVRKKGS
              610 620 630 640
EERRRPHKEE EEEAYYPPAP PPYSETDSQA SRERRLKKNL ALSRESLVV
LSR Isoform 2 (identifier: Q86X29-2)
(SEQ ID NO: 10)
                10 20 30 40 50
MQQDGLGVGT RNGSGKGRSV HPSWPWCAPR PLRYFGRDAR ARRAQTAAMA
                60 70 80 90 100
LAIQVTVSNP YHVVILFQPV TLPCTYQMTS TPTOPIVIWK YKSFCRDRIA
              110 120 130 140 150
DAFSPASVDN QLNAQLAAGN PGYNPYVECQ DSVRTVRVVA TKQGNAVTLG
              160 170 180 190 200
DYYQGRRITI TGNADLTFDQ TAWGDSGVYY CSVVSAQDLQ GNNEAYAELI
              210 220 230 240 250
VLGRTSGVAE LLPGFQAGPI EDWLFVVVVC LAAFLIFLLL GICWCQCCPH
              260 270 280 290 300
TCCCYVRCPC CPDKCCCPEA LYAAGKAATS GVPSIYAPST YAHLSPAKTP
              310 320 330 340 350
PPPAMIPMGP AYNGYPGGYP GDVDRSSSVR SGYRIQASQQ DDSMRVLYYM
              360 370 380 390 400
EKELANFDPS RPGPPSGPVE RAMSEVTSLH EDDWRSRPSR GPALTPIRDE
              410 420 430 440 450
EWGGHSPRSP RGWDQEPARE QAGGGWRARR PRARSVDALD DLTPPSTAES
              460 470 480 490 500
GSRSPTSNGG RSRAYMPPRS RSRDDLYDQD DSRDFPRSRD PHYDDFRSRE
              510 520 530 540 550
RPPADPRSHH HRTRDPRDNG SRSGDLPYDG RLLEEAVRKK GSEERRRPHK
              560 570 580 590
EEEERAYYPP APPPYSETDS QASRERRLKK NLALSRESLV V
LSR Isoform 3 (identifier Q86X29-3)
(SEQ ID NO: 11)
                10 20 30 40 50
MQQDGLGVGT RNGSGKGRSV HPSWPWCAPR PLRYFGRDAR ARRAQTAAMA
                60 70 80 90 100
LLAGGLSRGL GSHPAAAGRD AVVFVWLLLS TWCTAPARAI QVTVSNPYHV
              110 120 130 140 150
VILFQPVTLP CTYQMTSTPT QPIVIWKYKS FCRDRIADAF SPASVDNQLN
              160 170 180 190 200
AQLAAGNPGY NPYVECQDSV RTVRVVATKQ GNAVTLGDYY QGRRITITGN
              210 220 230 240 250
ADLTFDQTAW GDSGVYYCSV VSAQDLQGNN EAYAELIVLD WLFVVVVCLA
              260 270 280 290 300
AFLIFLLLGI CWCQCCPHTC CCYVRCPCCP DKCCCPEALY AAGKAATSGV
              310 320 330 340 350
PSIYAPSTYA HLSPAKTPPP PAMIPMGPAY NGYPGGYPGD VDRSSSAGGQ
              360 370 380 390 400
GSYVPLLRDT DSSVASVRSG YRIQASQQDD SMRVLYYMEK ELANFDPSRP
              410 420 430 440 450
GPPSGRVERA MSEVTSLHED DWRSRPSRGP ALTPIRDEEW GGHSPRSPRG
              460 470 480 490 500
WDQEPAREQA GGGWRARRPR ARSVDALDDL TPPSTAESGS RSPTSNGGRS
              510 520 530 540 550
RAYMPPRSRS RDDLYDQDDS RDFPRSRDPH YDDFRSRERP PADPRSHHHR
              560 570 580 590 600
TPDPRDNGSR SGDLPYDGRL LEEAVRKKGS EERRRPHKEE EEEAYYPPAP
              610 620
PPYSETDSQA SRERRIKKNL ALSRESLVV
LSR Isoform 4 (identifier: Q86X29-4)
(SEQ ID NO: 12)
                10 20 30 40 50
MQQDGLGVGT RNGSGKGRSV HPSWPWCAPR PLRYFGRDAR ARRAQTAAMA
                60 70 80 90 100
LLAGGLSRGL GSHPAAAGRD AVVFVWLLLS TWCTAPARAI QVTVSNPYHV
              110 120 130 140 150
VILFQPVTLP CTYQMTSTPT QPIVIWKYKS FCRDRIADAF SPASVDNQLN
              160 170 180 190 200
AQLAAGNPGY NPYVECQDSV RTVRVVATKQ GNAVTLGDYY QGRRITITGN
              210 220 230 240 250
ADLTFDQTAW GDSGVYYCSV VSAQDLQGNN EAYAELIVLD WLFVVVVCLA
              260 270 280 290 300
AFLIFLLLGI CWCQCCPHTC CCYVRCPCCP DKCCCPEALY AAGKAATSGV
              310 320 330 340 350
PSIYAPSTYA HLSPAKTPPP PAMIPMGPAY NGYPGGYPGD VDRSSSAGGQ
              360 370 380 390 400
GSYVPLLRDT DSSVASEVRS GYRIQASQQD DSMRVLYYME KELANFDPSR
              410 420 430 440 450
PGPPSGRVER AMSEVTSLHE DDWRSRPSRG PALTPIRDEE WGGHSPRSPR
              460 470 480 490 500
GWDQEPAREQ AGGGWRARRP RARSVDALDD LTPPSTAESG SRSPTSNGGR
              510 520 530 540 550
SRAYMPPRSR SRDDLYDQDD SRDFPRSRDP HYDDFRSRER PPADPRSHHH
              560 570 580 590 600
RTRDPRDNGS RSGDLPYDGR LLEEAVRKKG SEERRRPHKE EEEEAYYPPA
              610 620 630
PPPYSETDSQ ASRERRLKKN LALSRESLVV
LSR Isoform 5 (identifier: Q86X29-5)
(SEQ ID NO: 13)
                10 20 30 40 50
MQQDGLGVGT RNGSGKGRSV HPSWPWCAPR PLRYFGRDAR ARRAQTAAMA
                60 70 80 90 100
LLAGGLSRGL GSHPAAAGRD AVVFVWLLLS TWCTAPARAI QVTVSNPYHV
              110 120 130 140 150
VILFQPVTLP CTYQMTSTPT QPIVIWKYKS FCRDRIADAF SPASVDNQLN
              160 170 180 190 200
AQLAAGNPGY NPYVECQDSV RTVRVVATKQ GNAVTLGDYY QGRRITITGN
              210 220 230 240 250
ADLTFDQTAW GDSGVYYCSV VSAQDLQGNN EAYAELIVLV YAAGKAATSG
              260 270 280 290 300
VPSIYAPSTY AHLSPAKTPP PPAMIPMGPA YNGYPGGYPG DVDRSSSAGG
              310 320 330 340 350
QGSYVPLLRD TDSSVASEVR SGYRIQASQQ DDSMRVLYYM EKELANFDPS
              360 370 380 390 400
RPGPPSGRVE RAMSEVTSLH EDDWRSRPSR GPALTPIRDE EWGGHSPRSP
              410 420 430 440 450
RGWDQEPARE QAGGGWRARR PRARSVDALD DLTPPSTAES GSRSPTSNGG
              460 470 480 490 500
RSRAYMPPRS RSRDDLYDQD DSRDFPRSRD PHYDDFRSRE RPPADPRSHH
              510 520 530 540 550
HRTRDPRDNG SRSGDLPYDG RLLEEAVRKK GSEERRRPHK EEEEEAYYPP
              560 570 580
APPPYSETDS QASRERRLKK NLALSRESLV V
LSR Isoform 6 (identifier Q86X29-6)
(SEQ ID NO: 14)
                10 20 30 40 50
MALLAGGLSR GLGSHPAAAG RDAVVFVWLL LSTWCTAPAR AIQVTVSNPY
                60 70 80 90 100
HVVILFQPVT LPCTYQMTST PTQPIVIWKY KSFCRDRIAD AFSPASVDNQ
              110 120 130 140 150
LNAQLAAGNP GYNPYVECQD SVRTVRVVAT KQGNAVTLGD YYQGRRITIT
              160 170 180 190 200
GMYAAGKAAT SGVPSIYAPS TYAHLSPAKT PPPPAMIPMG PAYNGYPGGY
              210 220 230 240 250
PGDVDRSSSA GGQGSYVPLL RDTDSSVASE VRSGYRIQAS QQDDSMRVLY
              260 270 280 290 300
YMEKELANFD PSRPGPPSGR VERAMSEVTS LHEDDWRSRP SRGPALTPIR
              310 320 330 340 350
DEEWGGHSPR SPRGWDQEPA REQAGGGWRA RRPRARSVDA LDDLTPPSTA
              360 370 380 390 400
ESGSRSPTSN GGRSRAYMPP RSRSRDDLYD QDDSRDFPRS RDPHYDDFRS
              410 420 430 440 450
RERPPADPRS HHHRTRDPRD NGSRSGDLPY DGRLLEEAVR KKGSEERRRP
              460 470 480 490
HKEEEEEAYY PPAPPPYSET DSQASRERRL KKNLALSRES LVV
```

LSR proteins suitable to target for opening up the BBB can be any LSR protein known in the art, such as those in U.S. Pat. No. 9,409,987, incorporated herein by reference.

ILDR1 has 6 isoforms produced by alternative splicing.

```
ILDR1 isoform 1 (identifier: Q86SU0-1)
SEQ ID NO: 15
                10 20 30 40 50
MAWPKLPAPW LLLCTWLPAG CLSLLVTVQH TERYVTLFAS IILKCDYTTS
                60 70 80 90 100
AQLQDVVVTW RFKSFCKDPI FDYYSASYQA ALSLGQDPSN DCNDNQREVR
              110 120 130 140 150
IVAQRRGQNE PVLGVDYRQR KITIQNRADL VINEVMWWDH GVYYCTIEAP
              160 170 180 190 200
GDTSGDPDKE VKLIVLHWLT VIFIILGALL LLLLIGVCWC QCCPQYCCCY
              210 220 230 240 250
IRCPCCPAHC CCPEEALARH RYMKQAQALG PQMMGKPLYW GADRSSQVSS
              260 270 280 290 300
YPMHPLLQRD LSLPSSLPQM PMTQTTNQPP IANGVLEYLE KELRNLNLAQ
              310 320 330 340 350
PLPPDLKGRF GHPCSMLSSL GSEVVERRII HLPPLIRDLS SSRRTSDSLH
              360 370 380 390 400
QQWLTPIPSR PWDLREGRSH HHYPDFHQEL QDRGPKSWAL ERRELDPSWS
              410 420 430 440 450
GRHRSSRLNG SPIHWSDRDS LSDVPSSSEA RWRPSHPPFR SPCQERPRRP
              460 470 480 490 500
SPRESTQRHG RRRRHRSYSP PLPSGLSSWS SEEDKERQPQ SWRAHRRGSH
              510 520 530 540
SPHWPEEKPP SYRSLDITPG KNSRKKGSVE RRSEKDSSHS GPSVVI
ILDR1 isoform 2 (identifier: Q86SU0-2)
SEQ ID NO: 16
                10 20 30 40 50
MAWPKLPAPW LLLCTWLPAG CLSLLVTVQH TERYVTLFAS IILKCDYTTS
                60 70 80 90 100
AQLQDVVVTW RFKSFCKDPI FDYYSASYQA ALSLGQDPSN DCNDNQREVR
              110 120 130 140 150
IVAQRRGQNE PVLGVDYRQR KITIQNRADL VINEVMWWDH GVYYCTIEAP
              160 170 180 190 200
GDTSGDPDKE VKLIVLHWLT VIFIILGALL LLLLIGVCWC QCCPQYCCCY
              210 220 230 240 250
IRCPCCPAHC CCPEEDLSLP SSLPQMPMTQ TTNQPPIANG VLEYLEKELR
              260 270 280 290 300
NLNLAQPLPP DLKGRFGHPC SMLSSLGSEV VERRIIHLPP LIRDLSSSRR
              310 320 330 340 350
TSDSLHQQWL TPIPSRPWDL REGRSHHHYP DFHQELQDRG PKSWALERRE
              360 370 380 390 400
LDPSWSGRHR SSRLNGSPIH WSDRDSLSDV PSSSEARWRP SHPPFRSRCQ
              410 420 430 440 450
ERPRRPSPRE STQRHGRRRR HRSYSPPLPS GLSSWSSEED KERQPQSWRA
              460 470 480 490 500
HRRGSHSPHW PEEKPPSYRS LDITPGKNSR KKGSVERRSE KDSSHSGRSV
VI
ILDR1 isoform 3 (identifier: Q86SU0-3)
SEQ ID NO: 17
                10 20 30 40 50
MAWPKLPAPW LLLCTWLPAG CLSLLVTVQH TERYVTLFAS IILKCDYTTS
                60 70 80 90 100
AQLQDVVVTW RFKSFCKDPI FDYYSASYQA ALSLGQDPSN DCNDNQREVR
              110 120 130 140 150
IVAQRRGQNE PVLGVDYRQR KITIQNRADL VINEVMWWDH GVYYCTIEAP
              160 170 180 190 200
GDTSGDPDKE VKLIVLHWLT VIFIILGALL LLLLIGVCWC QCCPQYCCCY
              210 220 230 240 250
IRCPCCPAHC CCPEEALARH RYMKOAQALG PQMMGKPLYW GADRSSQVSS
              260
YPMHPLLQRA SRRCQ
ILDR1 isoform 4 (identifier: Q86SU0-4)
SEQ ID NO: 18
                10 20 30 40 50
MAWPKLPAPW LLLCTWLPAG CLSLLVTVQH TERYVTLFAS IIIKCDYTTS
                60 70 80 90 100
AQLQDVVVTW RFKSFCKDPI FDYYSASYQA ALSLGQDPSN DCCCPEEALA
              110 120 130 140 150
RHRYMKQAQA LGPQMMGKPL YWGADRSSQV SSYPMHPLLQ RDLSLPSSLP
              160 170 180 190 200
QMPMTQTTNQ PPIANGVLEY LEKELRNLNL AQPLPPDLKG RFGHPCSMLS
              210
SLGSENQIEE F
ILDR1 isoform 5 (identifier: Q865U0-5)
SEQ ID NO: 19
                10 20 30 40 50
MAWPKLPAPW LLLCTWLPAG CLSLLVTVQH TERYVTLFAS IILKCDYTTS
                60 70 80 90 100
AQLQDVVVTW RFKSFCKDPI FDYYSASYQA ALSLGQDPSN DCNDNQREVR
              110 120 130 140 150
IVAQRRGQNE PVLGVDYRQR KITIQNPLAR HRYMKQAQAL GPQMMGKPLY
              160 170 180 190 200
WGADRSSQVS SYPMHPLLQR DLSLPSSLPQ MPMTQTTNQP PIANGVLEYL
              210 220 230 240 250
EKELRNLNLA QPLPPDLKGR FGHPCSMLSS LGSEVVERRI IHLPPLIRDL
              260 270 280 290 300
SSSRRTSDSL HQQWLTPIPS RPWDLREGRS HHHYPDFHQE LQDRGPKSWA
              310 320 330 340 350
LERRELDPSW SGRHRSSRLN GSPIHWSDRD SLSDVPSSSE ARWRPSHPPF
              360 370 380 390 400
RSRCQERPRR PSPRESTQRH GRRRPHRSYS PPLPSGLSSW SSEEDKERQP
              410 420 430 440 450
QSWRAHRRGS HSPHWPEEKP PSYRSLDITP GKNSRKKGSV ERRSEKDSSH
SGRSVVI
ILDR1 isoform 6 (identifier: Q865U0-6)
(SEQ ID NO: 20)
                10 20 30 40 50
MAGNIFCPFA LFFLPMSRVG HLQHFLLLLA LGCLSLLVTV QHTERYVTLF
                60 70 80 90 100
ASIILKCDYT TSAQLQDVVV TWRFKSFCKD PIFDYYSASY QAALSLGQDP
              110 120 130 140 150
SNDCNDNQRE VRIVAQRRGQ NEPVLGVDYR QRKITIQNRA DLVINEVMWW
              160 170 180 190 200
DHGVYYCTIE APGDTSGDPD KEVKLIVLHW LTVIFIILGA LLLLLLIGVC
              210 220 230 240 250
WCQCCPQYCC CYIRCPCCPA HCCCPEEDLS LPSSLPQMPM TQTTNQPPIA
              260 270 280 290 300
NGVLEYLEKE LRNLNLAQPL PPDLKGRFGH PCSMLSSLGS EVVERRIIHL
              310 320 330 340 350
PPLIRDISSS RRTSDSLHQQ WLTPIPSRPW DLREGRSHHH YPDFHQELQD
              360 370 380 390 400
RGPKSWALER RELDPSWSGR HRSSRLNGSP IHWSDRDSLS DVPSSSEARW
              410 420 430 440 450
RPSHPPFRSR CQERPRRPSP RESTQRHGRR RRHRSYSPPL PSGLSSWSSE
              460 470 480 490 500
EDKERQPQSW RAHRRGSHSP HWPEEKPPSY RSLDITPGKN SRKKGSVERR
              510
SEKDSSHSGR SVVI
ILDR2 (identifier Q71H61-1)
(SEQ ID NO: 21)
                10 20 30 40 50
MDRVLLRWIS LFWLTAMVEG LQVTVPDKKK VAMLFQPTVL RCHFSTSSHQ
                60 70 80 90 100
PAVVQWKFKS YCQDRMGESL GMSSTRAQSL SKRNLEWDPY LDCLDSRRTV
              110 120 130 140 150
RVVASKQGST VTLGDFYRGR EITIVHDADL QIGKLMWGDS GLYYCIITTP
              160 170 180 190 200
DDLEGKNEDS VELLVLGRTG LLADLLPSFA VEIMPEWVFV GLVLLGVFLF
              210 220 230 240 250
FVLVGICWCQ CCPHSCCCYV RCPCCPDSCC CPQALYEAGK AAKAGYPPSV
              260 270 280 290 300
SGVPGPYSIP SVPLGGAPSS GMLMDKPHPP PLAPSDSTGG SHSVRKGYRI
              310 320 330 340 350
QADKERDSMK VLYYVEKELA QFDPARRMRG RYNNTISELS SLHEEDSNFR
              360 370 380 390 400
QSFHQMRSKQ FPVSGDLESN PDYNSGVMGG SSGASRGPSA MEYNKEDRES
              410 420 430 440 450
FRHSQPRSKS EMLSRKNFAT GVPAVSMDEL AAFADSYGQR PRRADGNSHE
              460 470 480 490 500
ARGGSRFERS ESRAHSGFYQ DDSLEEYYGQ RSRSREPLTD ADRGWAFSPA
              510 520 530 540 550
RRRPAEDAHL PRLVSRTPGT APKYDHSYLG SARERQARPE GASRGGSLET
              560 570 580 590 600
PSKRSAQLGP RSASYYAWSP PGTYKAGSSQ DDQEDASDDA LPPYSELELT
              610 620 630
RGPSYRGRDL PYHSNSEKKR KKEPAKKTND FPTRMSLVV
```

siRNA

One aspect of the present disclosure provides for a BBB opening agent comprising a synthetic siRNA.

Small (or short) interfering RNA (siRNA) can be used as a RNA interference (RNAi) tool for inducing short-term silencing of protein coding genes. siRNA is a synthetic RNA duplex designed to specifically target a particular mRNA for degradation.

As described herein, the siRNA can safely manipulate tricellular junction permeability. Furthermore, the siRNA administration is safe and can be administered systematically. The siRNA can open the tight junction in the blood brain barrier. The siRNA can be against angulin.

As described herein, siRNA has been generated to target angulin.

As described herein, a mixture of two siRNA can be used during injection to minimize non-specific effect while improving the target knockdown efficacy.

Inhibitory RNA techniques are methods that use engineered RNA molecules to inhibit gene expression. Various approaches, including the expression or injection of microRNA, short inhibiting RNA, double-stranded, or antisense RNA (e.g. morpholino oligomers), work via mechanisms that include transcript cleavage, sequestration and the inhibition of protein translation. Inhibitory RNA products and processes are well known; see e.g., Pecot et al. Nature Reviews Cancer 11, 59-67 (January 2011). Except as otherwise noted herein, therefore, the process of the present disclosure can be carried out in accordance with such processes.

siRNA targeting angulin can be human or mouse (e.g., SEQ ID NO: 1-SEQ ID NO: 8).

Human.
LSRh_siRNA_#216; sense sequence; 5′→3′
(SEQ ID NO: 1)
CTTCCAGAATGCAACAGGATT
LSRh_siRNA_#216; anti-sense sequence; 5′→3′
(SEQ ID NO: 2)
TCCTGTTGCATTCTGGAAGTT
LSRh_siRNA_#822; sense sequence; 5′→3′
(SEQ ID NO: 3)
ATGCTGACCTGACCTTTGATT
LSRh_siRNA_#822; anti-sense sequence; 5′→3′
(SEQ ID NO: 4)
TCAAAGGTCAGGTCAGCATTT
Mouse.
LSRm_siRNA_#736; sense sequence; 5′→3′
(SEQ ID NO: 5)
ATGCTGACCTGACCTTCGATT
LSRm_siRNA_#736; anti-sense sequence; 5′→3′
(SEQ ID NO: 6)
TCGAAGGTCAGGTCAGCATTT
LSRm_siRNA_#2119; sense sequence; 5′→3′
(SEQ ID NO: 7)
TTGGAATATTGATGAAACTTT
LSRm_siRNA_#2119; anti-sense sequence;5′→3′
(SEQ ID NO: 8)
AGTTTCATCAATATTCCAATT

Antibody

A BBB opening agent can comprise an antibody. The antibody can be an angulin antibody. The angulin antibody can open or manipulate the tight junction in the blood brain barrier. Antibodies can be those described in U.S. Pat. No. 8,415,455, US Pat Pub No. 2015/0190466, US Pat Pub No. 2014/0294765 and U.S. patent application Ser. No. 15/108,242 and are incorporated herein by reference. The antibody can be an antibody against a membrane receptor on the endothelium known as a transferrin receptor. The antibody can facilitate antibody mediated endocytosis crossing of the blood brain barrier. However, drugs endocytosed into the endothelium may be degraded by the endothelial cell.

Small Molecule

A BBB opening agent can comprise a small molecule. The small molecule can target angulin to open the tight junction in the blood brain barrier.

Making and using small molecule inhibitors of are well known; see e.g., Arkin et al., Small-molecule inhibitors of protein-protein interactions: progressing towards the dream, Nature Reviews Drug Discovery 3, 301-317 (April 2004)). Except as otherwise noted herein, therefore, the process of the present disclosure can be carried out in accordance with such processes.

Blood Brain Barrier (BBB)

An aspect of the present disclosure provides compositions and methods that can open up or manipulate the blood brain barrier (BBB) or make the BBB more permeable.

It is well known that a clear compartmentalization of the blood and the parenchyma within the brain is separated by a tight barrier—the blood brain barrier (BBB). The BBB has been shown to be a major impediment to targeted therapeutic delivery to the brain. It has been estimated that approximately 100% of large-molecule neurotherapeutics and over 95% of small-molecule drugs cannot penetrate the BBB due to presence of tight junction (TJ). Current approaches to circumvent the TJ are to employ the Trojan horse strategy to trigger receptor mediated transcytosis to facilitate drug delivery into the brain parenchyma. The strategy is employed via encapsulating therapeutics within liposome or nanoparticle carriers with antibody coating that target the endothelium's membrane surface receptors, such as transferrin or P-glycoprotein, to induce transcytosis. There are two intrinsic limitations of this transcellular approach: (1) receptor targeting can be very inefficient, relying upon random interaction between ligands and their cell surface receptors; and (2) transcytosis inevitably subjects drugs and their carriers to various intracellular metabolisms, which can delay, alter, or remove the drug effects.

The tight junction (TJ) in thin section electron microscopy has been shown to be composed of a series of direct membrane contacts. Freeze-fracture electron microscopy has shown the membrane protein interactions at the tight junction as a branching and anastomosing reticulum of “fibrils” or “strands” on the P fracture face. These fibrils have been demonstrated to be composed of integral membrane proteins directly involved in cell-cell interaction. The known integral membrane proteins of the tight junction include occludin, the Junctional Adhesion Molecules (JAMs), and the claudins. Regular bicellular tight junctions (bTJs) cannot practically seal some exceptional regions, namely tricellular tight junctions (tTJs), where the corners of three polygonal epithelial cells meet. Unlike the bTJ that contains ion channels of 4-7 Å in diameter, the tTJ is predicted to create a paracellular pathway with much larger diameter—˜10 nm surrounded by the central sealing element. As described herein, the proteins making the tTJ include tricellulin and angulins (LSR/angulin-1, ILDR1/angulin-2, and ILDR2/angulin-3).

The present disclosure provides for a method to regulate the tTJ permeability by combining siRNAs against the LSR (angulin-1) gene with temozolomide or doxorubicin into intravenous injections and proves that such a method can significantly increase the permeability of temozolomide or doxorubicin across the BBB into the brain parenchyma. The present disclosure presents a new route to deliver important cancer drugs into brain parenchyma to treat brain tumors that are normally unreachable due to the blood brain barrier.

As described herein, siRNA molecules can be used to open the tight junction in order to deliver drugs (e.g., cancer drugs) into the brain parenchyma. For example, there are many effective cancer drugs available but unfortunately none of them work for brain tumor due to the presence of blood brain barrier.

The compositions and methods as described herein have been shown to be safely and transiently open the blood brain barrier. Furthermore, the compositions and methods as described herein do not cause global tight junction breakdown in the brain or lead to neuroinflammation.

The present disclosure provides a safe, feasible, and controllable means to open the blood brain barrier to allow drugs (e.g., cancer drugs) to reach the tumor cells and dramatically improve the prognosis of brain tumor patients by manipulating tricellular tight junction permeability. As described herein, manipulating the tricellular tight junction has been shown to be safe and does not affect normal BBB function, such as preventing T cell penetration.

Processes characterizing the BBB are well known; see e.g., Zhao et al. 2015 Cell (163) 1064-1078. Except as otherwise noted herein, therefore, the process of the present disclosure can be carried out in accordance with such processes.

Processes for isolating the brain endothelial cells from biopsies or mouse whole brains are well known. A unique property of the endothelial cell is utilized, which is that the endothelial cells are very resistant to an antibiotic known as puromycin. A brain sample can be broken down into individual cells when puromycin is added. Most neuron and glial cells will die at low dose of puromycin, leaving behind endothelial cells as the only surviving ones.

Therapeutic Agents

The present disclosure provides for a compositions and methods of treatment that can dramatically improve the prognosis and survival rate of patients (e.g., brain tumor patients) due to the described transient opening the blood brain barrier to allow a therapeutic agent (such as a cancer drug, to reach the tumor cells) which was previously not possible even for small molecule drugs such as temozolomide (194 Da) or doxorubicin (544 Da). As such, the methods as described herein can be used in combination with any therapeutic agent (e.g., a neurotherapeutic) that can treat a pathology of the brain that needs to cross the BBB. A brain pathology can be, for example, a brain cancer, stroke, or a neurodegenerative disease.

There are many effective cancer drugs available, but do not work for a brain tumor due to the presence of the blood brain barrier. One aspect of the present disclosure provides for manipulation of the tricellular tight junction in the BBB to allow for the permeation of these cancer drugs into the brain. A number of chemotherapy agents have been approved including temozolomide (194 Da). However, due the presence of blood brain barrier, even small molecule drugs such as temozolomide can barely reach the tumor cells and only extend the lifespan of patients by 3-4 months. Other more effective cancer drugs such as doxorubicin (with a molecular weight of 544 Da) were completely impermeable to the blood brain barrier, thus unable to kill the tumor cells.

As an example, a therapeutic agent can comprise a chemotherapeutic agent that can be used in combination with the BBB opening agent. The chemotherapeutic agent can be one or more selected from the group consisting of: Abiraterone Acetate; Abitrexate (Methotrexate); Abraxane (Paditaxel Albumin-stabilized Nanoparticle Formulation); ABVD; ABVE; ABVE-PC; AC; AC-T; Adcetris (Brentuximab Vedotin); ADE; Ado-Trastuzumab Emtansine; Adriamycin (Doxorubicin Hydrochloride); Afatinib Dimaleate; Afinitor (Everolimus); Akynzeo (Netupitant and Palonosetron Hydrochloride); Aldara (Imiquimod); Aldesleukin; Alecensa (Alectinib); Alectinib; Alemtuzumab: Alkeran for Injection (Melphalan Hydrochloride); Alkeran Tablets (Melphalan); Alimta (Pemetrexed Disodium); Aloxi (Palonosetron Hydrochloride); Ambochlorin (Chlorambucil); Amboclorin (Chlorambucil); Amifostine; Aminolevulinic Acid; Anastrozole; Aprepitant; Aredia (Pamidronate Disodium); Arimidex (Anastrozole); Aromasin (Exemestane); Arranon (Nelarabine); Arsenic Trioxide; Arzerra (Ofatumumab); Asparaginase Erwinia chrysanthemi; Atezolizumab; Avastin (Bevacizumab); Avelumab; Axitinib; Azacitidine; Bavencio (Avelumab); BEACOPP; Becenum (Carmustine); Beleodaq (Belinostat); Belinostat; Bendamustine Hydrochloride; BEP; Bevacizumab; Bexarotene; Bexxar (Tositumomab and Iodine I 131 Tositumomab); Bicalutamide; BiCNU (Carmustine); Bleomycin; Blinatumomab; Blincyto (Blinatumomab); Bortezomib; Bosulif (Bosutinib); Bosutinib; Brentuximab Vedotin; BuMel; Busulfan; Busulfex (Busulfan); Cabazitaxel; Cabometyx (Cabozantinib-S-Malate); Cabozantinib-S-Malate; CAF; Campath (Alemtuzumab); Camptosar (Irinotecan Hydrochloride); Capecitabine; CAPOX; Carac (Fluorouracil); Carboplatin; CARBOPLATIN-TAXOL; Carfilzomib; Carmubris (Carmustine); Carmustine; Carmustine Implant; Casodex (Bicalutamide); CEM; Ceritinib; Cerubidine (Daunorubicin Hydrochloride); Cervarix (Recombinant HPV Bivalent Vaccine); Cetuximab; CEV; Chlorambucil; CHLORAMBUCIL-PREDNISONE; CHOP; Cisplatin; Cladribine; Clafen (Cyclophosphamide); Clofarabine; Clofarex (Clofarabine); Clolar (Clofarabine); CMF; Cobimetinib; Cometriq (Cabozantinib-S-Malate); COPDAC; COPP; COPP-ABV; Cosmegen (Dactinomycin); Cotellic (Cobimetinib); Crizotinib; CVP; Cyclophosphamide; Cyfos (Ifosfamide); Cyramza (Ramucirumab); Cytarabine; Cytarabine Liposome; Cytosar-U (Cytarabine); Cytoxan (Cyclophosphamide); Dabrafenib; Dacarbazine; Dacogen (Decitabine); Dactinomycin; Daratumumab; Darzalex (Daratumumab); Dasatinib; Daunorubicin Hydrochloride; Decitabine; Defibrotide Sodium; Defitelio (Defibrotide Sodium); Degarelix; Denileukin Diftitox; Denosumab; DepoCyt (Cytarabine Liposome); Dexamethasone; Dexrazoxane Hydrochloride; Dinutuximab; Docetaxel; Doxil (Doxorubicin Hydrochloride Liposome); Doxorubicin Hydrochloride; Doxorubicin Hydrochloride Liposome; Dox-SL (Doxorubicin Hydrochloride Liposome); DTIC-Dome (Dacarbazine); Efudex (Fluorouracil); Elitek (Rasburicase); Ellence (Epirubicin Hydrochloride); Elotuzumab; Eloxatin (Oxaliplatin); Eltrombopag Olamine; Emend (Aprepitant); Empliciti (Elotuzumab); Enzalutamide; Epirubicin Hydrochloride; EPOCH; Erbitux (Cetuximab); Eribulin Mesylate; Erivedge (Vismodegib); Erlotinib Hydrochloride; Erwinaze (Asparaginase Erwinia chrysanthemi); Ethyol (Amifostine); Etopophos (Etoposide Phosphate); Etoposide; Etoposide Phosphate; Evacet (Doxorubicin Hydrochloride Liposome); Everolimus; Evista (Raloxifene Hydrochloride); Evomela (Melphalan Hydrochloride); Exemestane; 5-FU (Fluorouracil Injection); 5-FU (Fluorouracil); Fareston (Toremifene); Farydak (Panobinostat); Faslodex (Fulvestrant); FEC; Femara (Letrozole); Filgrastim; Fludara (Fludarabine Phosphate); Fludarabine Phosphate; Fluoroplex (Fluorouracil); Fluorouracil Injection; Fluorouracil; Flutamide; Folex (Methotrexate); Folex PFS (Methotrexate); FOLFIRI; FOLFIRI-BEVACIZUMAB; FOLFIRI-CETUXIMAB; FOLFIRINOX; FOLFOX; Folotyn (Pralatrexate); FU-LV; Fulvestrant; Gardasil (Recombinant HPV Quadrivalent Vaccine); Gardasil 9 (Recombinant HPV Nonavalent Vaccine); Gazyva (Obinutuzumab); Gefitinib; Gemcitabine Hydrochloride; Gemcitabine-Cisplatin; Gemcitabine-Oxaliplatin; Gemtuzumab Ozogamicin; Gemzar (Gemcitabine Hydrochloride); Gilotrif (Afatinib Dimaleate); Gleevec (Imatinib Mesylate); Gliadel (Carmustine Implant); Gliadel wafer (Carmustine Implant); Glucarpidase; Goserelin Acetate; Halaven (Eribulin Mesylate); Hemangeol (Propranolol Hydrochloride); Herceptin (Trastuzumab); HPV Bivalent Vaccine, Recombinant; HPV Nonavalent Vaccine, Recombinant; HPV Quadrivalent Vaccine, Recombinant; Hycamtin (Topotecan Hydrochloride); Hydrea (Hydroxyurea); Hydroxyurea; Hyper-CVAD; Ibrance (Palbocidib); Ibritumomab Tiuxetan; Ibrutinib; ICE; Idusig (Ponatinib Hydrochloride); Idamycin (Idarubicin Hydrochloride); Idarubicin Hydrochloride; Idelalisib; Ifex (Ifosfamide); Ifosfamide; Ifosfamidum (Ifosfamide); IL-2 (Aldesleukin); Imatinib Mesylate; Imbruvica (Ibrutinib); Imiquimod; Imlygic (Talimogene Laherparepvec); Inlyta (Axitinib); Interferon Alfa-2b, Recombinant; Interleukin-2 (Aldesleukin); Intron A (Recombinant Interferon Alfa-2b); Iodine I 131 Tositumomab and Tositumomab; Ipilimumab; Iressa (Gefitinib); Irinotecan Hydrochloride; Irinotecan Hydrochloride Liposome; Istodax (Romidepsin); Ixabepilone; Ixazomib Citrate; Ixempra (Ixabepilone); Jakafi (Ruxolitinib Phosphate); JEB; Jevtana (Cabazitaxel); Kadcyla (Ado-Trastuzumab Emtansine); Keoxifene (Raloxifene Hydrochloride); Kepivance (Palifermin); Keytruda (Pembrolizumab); Kisqali (Ribociclib); Kyprolis (Carfilzomib); Lanreotide Acetate; Lapatinib Ditosylate; Lartruvo (Olaratumab); Lenalidomide; Lenvatinib Mesylate; Lenvima (Lenvatinib Mesylate); Letrozole; Leucovorin Calcium; Leukeran (Chlorambucil); Leuprolide Acetate; Leustatin (Cladribine); Levulan (Aminolevulinic Acid); Linfolizin (Chlorambucil); LipoDox (Doxorubicin Hydrochloride Liposome); Lomustine; Lonsurf (Trifluridine and Tipiracil Hydrochloride); Lupron (Leuprolide Acetate); Lupron Depot (Leuprolide Acetate); Lupron Depot-Ped (Leuprolide Acetate); Lynparza (Olaparib); Marqibo (Vincristine Sulfate Liposome); Matulane (Procarbazine Hydrochloride); Mechlorethamine Hydrochloride; Megestrol Acetate; Mekinist (Trametinib); Melphalan; Melphalan Hydrochloride; Mercaptopurine; Mesna; Mesnex (Mesna); Methazolastone (Temozolomide); Methotrexate; Methotrexate LPF (Methotrexate); Methylnaltrexone Bromide; Mexate (Methotrexate); Mexate-AQ (Methotrexate); Mitomycin C; Mitoxantrone Hydrochloride; Mitozytrex (Mitomycin C); MOPP; Mozobil (Plerixafor); Mustargen (Mechlorethamine Hydrochloride); Mutamycin (Mitomycin C); Myleran (Busulfan); Mylosar (Azacitidine); Mylotarg (Gemtuzumab Ozogamicin); Nanoparticle Paclitaxel (Paclitaxel Albumin-stabilized Nanoparticle Formulation); Navelbine (Vinorelbine Tartrate); Necitumumab; Nelarabine; Neosar (Cyclophosphamide); Netupitant and Palonosetron Hydrochloride; Neulasta (Pegfilgrastim); Neupogen (Filgrastim); Nexavar (Sorafenib Tosylate); Nilandron (Nilutamide); Nilotinib; Nilutamide; Ninlaro (Ixazomib Citrate); Nivolumab; Nolvadex (Tamoxifen Citrate); Nplate (Romiplostim); Obinutuzumab; Odomzo (Sonidegib); OEPA; Ofatumumab; OFF; Olaparib; Olaratumab; Omacetaxine Mepesuccinate; Oncaspar (Pegaspargase); Ondansetron Hydrochloride; Onivyde (Irinotecan Hydrochloride Liposome); Ontak (Denileukin Diftitox); Opdivo (Nivolumab); OPPA; Osimertinib; Oxaliplatin; Paditaxel; Paclitaxel Albumin-stabilized Nanoparticle Formulation; PAD; Palbocidib; Palifermin; Palonosetron Hydrochloride; Palonosetron Hydrochloride and Netupitant; Pamidronate Disodium; Panitumumab; Panobinostat; Paraplat (Carboplatin); Paraplatin (Carboplatin); Pazopanib Hydrochloride; PCV; PEB; Pegaspargase; Pegfilgrastim; Peginterferon Alfa-2b; PEG-Intron (Peginterferon Alfa-2b); Pembrolizumab; Pemetrexed Disodium; Perjeta (Pertuzumab); Pertuzumab; Platinol (Cisplatin); Platinol-AQ (Cisplatin); Plerixafor; Pomalidomide; Pomalyst (Pomalidomide); Ponatinib Hydrochloride; Portrazza (Necitumumab); Pralatrexate; Prednisone; Procarbazine Hydrochloride; Proleukin (Aldesleukin); Prolia (Denosumab); Promacta (Eltrombopag Olamine); Propranolol Hydrochloride; Provenge (Sipuleucel-T); Purinethol (Mercaptopurine); Purixan (Mercaptopurine); Radium 223 Dichloride; Raloxifene Hydrochloride; Ramucirumab; Rasburicase; R-CHOP; R-CVP; Recombinant Human Papillomavirus (HPV) Bivalent Vaccine; Recombinant Human Papillomavirus (HPV) Nonavalent Vaccine; Recombinant Human Papillomavirus (HPV) Quadrivalent Vaccine; Recombinant Interferon Alfa-2b; Regorafenib; Relistor (Methylnaltrexone Bromide); R-EPOCH; Revlimid (Lenalidomide); Rheumatrex (Methotrexate); Ribociclib; R-ICE; Rituxan (Rituximab); Rituximab; Rolapitant Hydrochloride; Romidepsin; Romiplostim; Rubidomycin (Daunorubicin Hydrochloride); Rubraca (Rucaparib Camsylate); Rucaparib Camsylate; Ruxolitinib Phosphate; Sclerosol Intrapleural Aerosol (Talc); Siltuximab; Sipuleucel-T; Somatuline Depot (Lanreotide Acetate); Sonidegib; Sorafenib Tosylate; Sprycel (Dasatinib); STANFORD V; Sterile Talc Powder (Talc); Steritalc (Talc); Stivarga (Regorafenib); Sunitinib Malate; Sutent (Sunitinib Malate); Sylatron (Peginterferon Alfa-2b); Sylvant (Siltuximab); Synribo (Omacetaxine Mepesuccinate); Tabloid (Thioguanine); TAC; Tafinlar (Dabrafenib); Tagrisso (Osimertinib); Talc; Talimogene Laherparepvec; Tamoxifen Citrate; Tarabine PFS (Cytarabine); Tarceva (Erlotinib Hydrochloride); Targretin (Bexarotene); Tasigna (Nilotinib); Taxol (Paclitaxel); Taxotere (Docetaxel); Tecentriq (Atezolizumab); Temodar (Temozolomide); Temozolomide; Temsirolimus; Thalidomide; Thalomid (Thalidomide); Thioguanine; Thiotepa; Tolak (Fluorouracil); Topotecan Hydrochloride; Toremifene; Torisel (Temsirolimus); Tositumomab and Iodine I 131 Tositumomab; Totect (Dexrazoxane Hydrochloride); TPF; Trabectedin; Trametinib; Trastuzumab; Treanda (Bendamustine Hydrochloride); Trifluridine and Tipiracil Hydrochloride; Trisenox (Arsenic Trioxide); Tykerb (Lapatinib Ditosylate); Unituxin (Dinutuximab); Uridine Triacetate; VAC; Vandetanib; VAMP; Varubi (Rolapitant Hydrochloride); Vectibix (Panitumumab); VelP; Velban (Vinblastine Sulfate); Velcade (Bortezomib); Velsar (Vinblastine Sulfate); Vemurafenib; Venclexta (Venetoclax); Venetoclax; Viadur (Leuprolide Acetate); Vidaza (Azacitidine); Vinblastine Sulfate; Vincasar PFS (Vincristine Sulfate); Vincristine Sulfate; Vincristine Sulfate Liposome; Vinorelbine Tartrate; VIP; Vismodegib; Vistogard (Uridine Triacetate); Voraxaze (Glucarpidase); Vorinostat; Votrient (Pazopanib Hydrochloride); Wellcovorin (Leucovorin Calcium); Xalkori (Crizotinib); Xeloda (Capecitabine); XELIRI; XELOX; Xgeva (Denosumab); Xofigo (Radium 223 Dichloride); Xtandi (Enzalutamide); Yervoy (Ipilimumab); Yondelis (Trabectedin); Zaltrap (Ziv-Aflibercept); Zarxio (Filgrastim); Zelboraf (Vemurafenib); Zevalin (Ibritumomab Tiuxetan); Zinecard (Dexrazoxane Hydrochloride); Ziv-Aflibercept; Zofran (Ondansetron Hydrochloride); Zoladex (Goserelin Acetate); Zoledronic Acid; Zolinza (Vorinostat); Zometa (Zoledronic Acid); Zydelig (Idelalisib); Zykadia (Ceritinib); or Zytiga (Abiraterone Acetate).

As another example, a therapeutic agent can comprise a stroke drug that can be used in combination with the BBB opening agent. The stroke drug can be one or more selected from the group consisting of: a thrombolytic agent, an anticonvulsant agent, an anti-platelet agent, an anti-coagulant agent or a hematologic agent, an analgesic, a beta blocker or alpha activity agent, an ACE inhibitor, a calcium channel blocker, a vasodilator, a cholesterol-lowering and blood-pressure-lowering medicine, a blood pressure medicine, or medicines used to treat depression and pain. An anticoagulant can be warfarin (for example, Coumadin, Jantoven), Dabigatran (Pradaxa), Rivaroxaban (Xarelto), Apixaban (Eliquis), or Edoxaban (Savaysa). A thrombolytic can be an IV tissue plasminogen activator (TPA) or Alteplase (Activase). An antiplatelet medication can be Aspirin (for example, Bayer), aspirin combined with dipyridamole (Aggrenox) is a safe and effective alternative to aspirin, or Clopidogrel (Plavix). A cholesterol-lowering and blood-pressure-lowering medicines can be a statin, angiotensin II receptor blockers (ARBs), angiotensin-converting enzyme (ACE) inhibitors, beta-blockers, calcium channel blockers, or diuretics. Medicines used to treat depression and pain can be amitriptyline, bupropion (Wellbutrin), citalopram (Celexa), fluoxetine (Prozac), sertraline (Zoloft), venlafaxine (Effexor). An anticonvulsant can be Diazepam (Valium) or Lorazepam (Ativan). An analgesic can be acetaminophen (Tylenol, Feverall, Aspirin Free Anacin). A beta blocker or alpha activity medication can be Labetalol (Normodyne, Trandate). An ACE Inhibitor can be Enalapril (Vasotec). A calcium channel blockers can be Nicardipine (Cardene). A vasodilator can be Nitroprusside sodium (Nipride, Nitropress, Sodium Nitroprusside).

As another example, a therapeutic agent can comprise a neurodegenerative disease drug or an Alzheimer's drug that can be used in combination with the BBB opening agent. The neurodegenerative disease drug or an Alzheimer's drug can be one or more selected from the group consisting of: cholinesterase inhibitors (Aricept, Exelon, Razadyne); memantine (Namenda); donepezil (Aricept); galantamine (Razadyne); memantine (Namenda); rivastigmine (Exelon); memantine+donepezil (Namzaric); ergoloid, Vitamin E, Alpha E, hydergine, Aqua-E, Aqua Gem-E, Aquasol E, Aquavite-E, E-400 clear, E-600, E-Gems, ergoloid mesylates, or etanercept.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

A nucleic acid sequence or amino acid sequence (e.g., DNA, RNA, a genetic sequence, polynucleotide, oligonucleotide, primer, protein, polypeptide, peptide) can have about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; or about 99% sequence identity to a reference sequence or a naturally occurring sequence or contain at least one substitution modification to the reference sequence or naturally occurring sequence. Recitation of each of these discrete values is understood to include ranges between each value.

A nucleic acid sequence or an amino acid sequence can be operably linked to a heterologous promoter.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, GIn by Asn, Val by lie, Leu by lie, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀ [Na⁺])+0.41 (fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoom and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of opening up or manipulating the BBB to make the BBB more permeable for treating a brain pathology, neurological disease, disorder, or condition (e.g., cancer, stroke, neurodegenerative disease, Alzheimer's disease) in a subject in need administration of a therapeutically effective amount of blood brain barrier opening agent (BBB opening agent), so as to open the BBB or make the BBB permeable.

Methods described herein are generally performed on a subject in need thereof. A subject in need of the therapeutic methods described herein can be a subject having, diagnosed with, suspected of having, or at risk for developing a brain pathology or neurological disease, disorder, or condition. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of BBB opening agent is, for example, that amount that would cause the desired therapeutic effect in a subject while minimizing undesired side effects. In various embodiments, an effective amount of BBB opening agent described herein can substantially inhibit a brain pathology or neurological disease, disorder, or condition, slow the progress of brain pathology or a neurological disease, disorder, or condition, or limit the development of a brain pathology or neurological disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

According to the methods described herein, administration can be systemic, enteral, or parenteral.

When used in the treatments described herein, a therapeutically effective amount of BBB opening agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to open the BBB or make the BBB permeable.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of BBB opening agent can occur as a single event or over a time course of treatment. For example, BBB opening agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a neurological disease, disorder, or condition.

A BBB opening agent can be administered simultaneously or sequentially with another agent used to treat a brain or spinal cord tumor, brain or spinal cord cancer, or a neurological disease disorder, or condition, such as a chemotherapeutic agent, a neurotherapeutic drug, or another agent. For example, a BBB opening agent can be administered simultaneously with another agent, such as a chemotherapeutic agent, a neurotherapeutic drug, or another agent used to treat a brain or spinal cord tumor, brain or spinal cord cancer, or a neurological disease disorder, or condition. Simultaneous administration can occur through administration of separate compositions, each containing one or more of a BBB opening agent, a chemotherapeutic agent, a neurotherapeutic drug, or another agent. Simultaneous administration can occur through administration of one composition containing two or more of a BBB opening agent, a chemotherapeutic agent, a neurological drug, or another agent. A BBB opening agent can be administered sequentially with a chemotherapeutic agent, a neurotherapeutic drug, or another agent. For example, a BBB opening agent can be administered before or after administration of a chemotherapeutic agent, a neurotherapeutic drug, or another agent.

As an example, a chemotherapeutic agent can be temozolomide or doxorubicin.

A BBB opening agent can be administered simultaneously or sequentially with another agent, such as an antibiotic, an anti-inflammatory, or another agent. For example, another agent can be any drug used to treat a brain or spinal cord tumor, brain or spinal cord cancer, or a neurological disease disorder, or condition. For example, a BBB opening agent can be administered simultaneously with another agent, such as an antibiotic or an anti-inflammatory. Simultaneous administration can occur through administration of separate compositions, each containing one or more of a BBB opening agent, an antibiotic, an anti-inflammatory, or another agent. Simultaneous administration can occur through administration of one composition containing two or more of a BBB opening agent, an antibiotic, an anti-inflammatory, or another agent. A BBB opening agent can be administered sequentially with an antibiotic, an anti-inflammatory, or another agent. For example, a BBB opening agent can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening blood brain barrier agents against angulin.

The disclosed methods find use in the screening of a variety of different candidate molecules (e.g., potentially candidate BBB opening agent molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a blood brain barrier opening agent, optionally in combination with a pharmaceutical/therapeutic agent comprising, for example, a chemotherapeutic agent, Alzheimer's drug, or stroke drug. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Overview of Tight Junction Biology Studies

The following example describes an overview of the studies of the tight junction biology in the blood brain barrier (BBB).

The studies of the tight junction biology of the blood brain barrier have established the following: (1) tricellular tight junction has different permeability profiles compared to bicellular tight junction; (2) tricellular tight junction is responsible for large size organic molecule permeation while bicellular tight junction is for small size inorganic molecules such as ions; (3) deletion of the angulin protein from tricellular tight junction increases permeability of large molecules such as cancer drugs, temozolomide (194 Da) and doxorubicin (544 Da); (4) deletion of angulin protein did not affect the bicellular tight junction function or the overall TJ barrier structure; and (5) deletion of the angulin protein increases the temozolomide and doxorubicin permeability to cerebral cortex in live mice in vivo.

The studies of the tight junction biology of the blood brain barrier, as described herein, demonstrate that a novel genetic pathway can be manipulated via intravenous siRNA injection to transiently open the blood brain barrier to allow cancer drugs to permeate through the paracellular space to the brain parenchyma.

As described herein, the approach has been tested in mouse models in vivo and it was then demonstrated that knock-down of the tight junction gene can allow significant increase of permeability of temozolomide (194 Da) and doxorubicin (544 Da) through the paracellular space to the brain parenchyma. Furthermore, the siRNA injection does not cause global tight junction breakdown in the brain or lead to neuroinflammation in tested mouse models.

Based upon these discoveries, it has been shown that manipulating tricellular tight junction permeability can present a safe, feasible, and controllable means to open the blood brain barrier to allow cancer drugs to reach the tumor cells and dramatically improve the prognosis of brain tumor patients.

Design of siRNA Against Angulin Gene

siRNA was designed against the angulin gene. The synthesis of siRNA molecule is straightforward (with current RNA oligo synthesizer). The synthetic siRNA is 21 nucleotide long.

The following are the siRNA sequences for a human and a mouse LSR gene.

Human.
LSRh_siRNA_#216; sense sequence; 5′→3′
(SEQ ID NO: 1)
CTTCCAGAATGCAACAGGATT
LSRh_siRNA_#216; anti-sense sequence; 5′→3′
(SEQ ID NO: 2)
TCCTGTTGCATTCTGGAAGTT
LSRh_siRNA_#822; sense sequence; 5′→3′
(SEQ ID NO: 3)
ATGCTGACCTGACCTTTGATT
LSRh_siRNA_#822; anti-sense sequence; 5′→3′
(SEQ ID NO: 4)
TCAAAGGTCAGGTCAGCATTT
Mouse.
LSRm_siRNA_#736; sense sequence; 5′→3′
(SEQ ID NO: 5)
ATGCTGACCTGACCTTCGATT
LSRm_siRNA_#736; anti-sense sequence; 5′→3′
(SEQ ID NO: 6)
TCGAAGGTCAGGTCAGCATTT
LSRm_siRNA_#2119; sense sequence; 5′→3′
(SEQ ID NO: 7)
TTGGAATATTGATGAAACTTT
LSRm_siRNA_#2119; anti-sense sequence;5′→3′
(SEQ ID NO: 8)
AGTTTCATCAATATTCCAATT

The following examples describe the in vivo animal studies showing the safe intravenous injection of siRNA in mice and demonstrated that the siRNA can open the blood brain barrier. The following examples also describe the animal studies showing that injecting siRNA against angulin gene can open the tricellular junction. The following examples further describe the animal studies showing that injecting siRNA against angulin gene can increases temozolomide and doxorubicin permeability to the brain.

Method of Producing siRNA

Single stranded sense and antisense RNA molecules were chemically synthesized by automated solid phase oligonucleotide synthesizer (Integrated DNA Technologies). Equal moles of sense and antisense RNA molecules were mixed to generate a functional siRNA duplex molecule. Such an siRNA duplex molecule was then mixed with the in vivo-jetPEI (liposome) reagent to form an in vivo grade siRNA molecule.

Materials

1. In vivo-jetPEI siRNA delivery reagent (Polyplus-transfection)

2. Lipofectamine 2000 transfection reagent (ThermoFisher)

3. Fluorescein (Sigma Aldrich)

4. Fluorescein-Temozolomide Conjugate (synthesized by CellMosaic Inc)

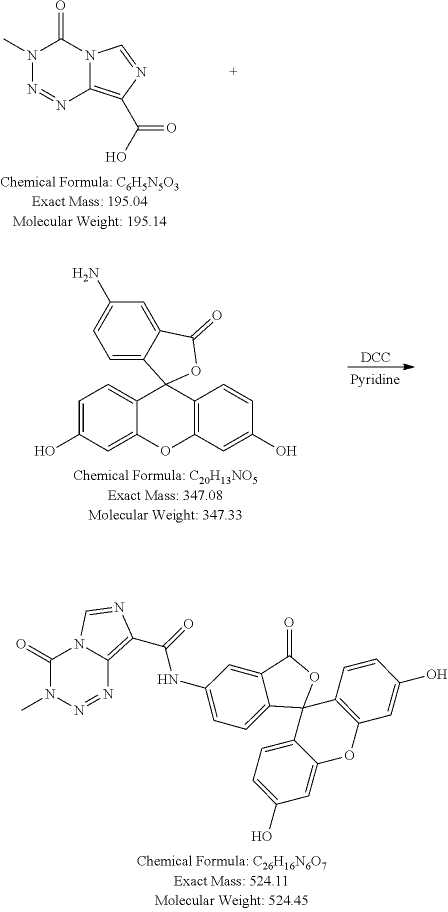 [SMILES: Cn1nnc2c(C(=O)O)ncn2c1=O ; "+" ; Nc1ccc2c(c1)C(=O)OC21c2ccc(O)cc2Oc2cc(O)ccc21 ; "DCC" ; Cn1nnc2c(C(=O)Nc3ccc4c(c3)C(=O)OC43c4ccc(O)cc4Oc4cc(O)ccc43)ncn2c1=O]

5. Fluorescein-doxorubicin Conjugate (synthesized by CellMosaic Inc)

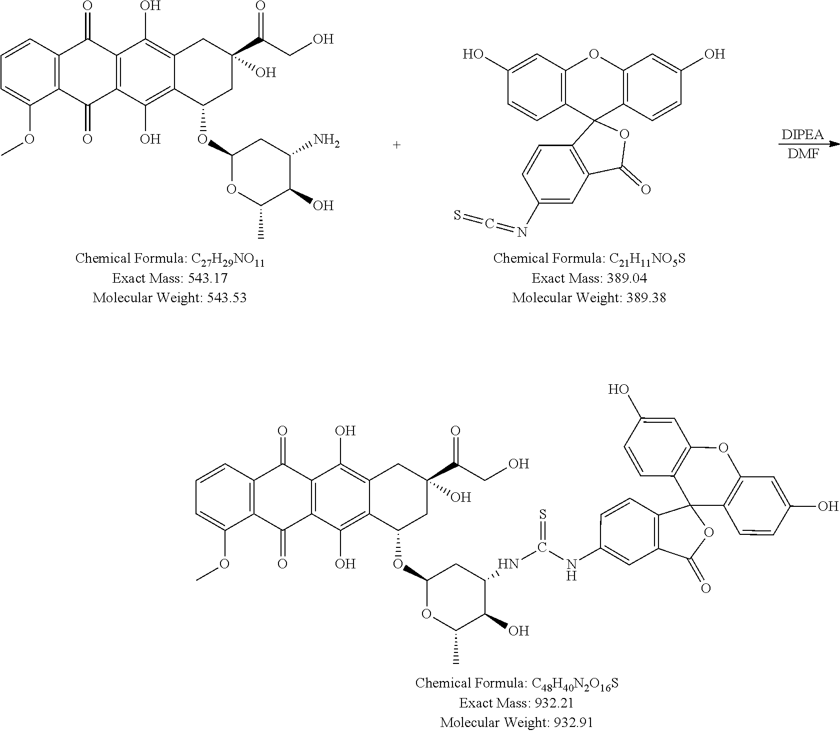

### Example 2: In Vivo Knockdown of LSR mRNA Levels in the Mouse Brain

The following example describes the in vivo knockdown of LSR mRNA levels in the mouse brain.

Approach:

1.5 mg/kg BW-1 of mouse LSRm_siRNA_#736 and 1.5 mg/kg BW-1 of LSRm_siRNA_#2119 were mixed and injected with in vivo-jetPEI delivery reagent into mouse tail vein on day 0. On day 1, another dose of 1.5 mg/kg BW-1 of mouse LSRm_siRNA_#736 and 1.5 mg/kg BW-1 of LSRm_siRNA_#2119 was injected with in vivo-jetPEI delivery reagent into the same mouse tail vein to boost the effects. On day two, mouse cerebral cortex was dissected out and assayed by quantitative PCR for changes in LSR mRNA levels.

Results:

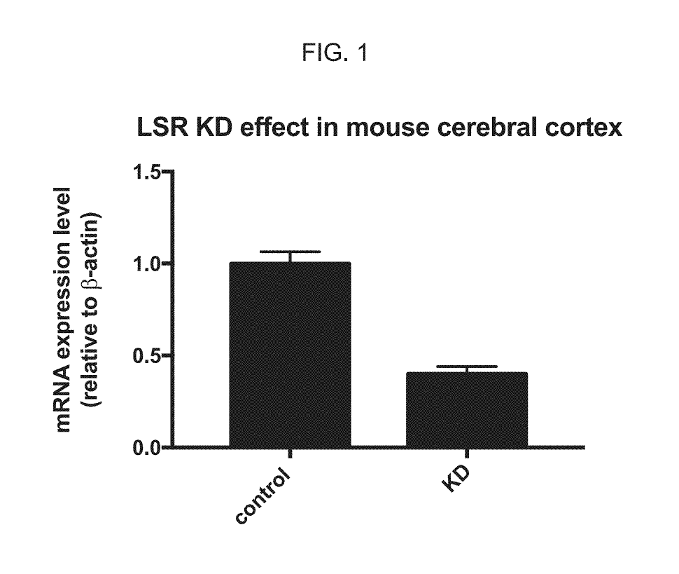

N=4 mice receiving LSR siRNA injection (KD) (siRNA #736+#2119); N=4 mice receiving scrambled siRNA injection (Control) (see e.g., FIG. 1). LSR siRNA injection significantly reduced the LSR cerebral expression by 60% (p<0.05).

### Example 3: In Vivo Knockdown of LSR Protein Levels in the Mouse Brain

The following example describes the in vivo knockdown of LSR protein levels in the mouse brain.

Approach:

1.5 mg/kg BW-1 of mouse LSRm_siRNA_#736 and 1.5 mg/kg BW-1 of LSRm_siRNA_#2119 were mixed and injected with in vivo-jetPEI delivery reagent into mouse tail vein on day 0. On day 1, another dose of 1.5 mg/kg BW-1 of mouse LSRm_siRNA_#736 and 1.5 mg/kg BW-1 of LSRm_siRNA_#2119 was injected with in vivo-jetPEI delivery reagent into the same mouse tail vein to boost the effects. On day two, mouse cerebral cortex was dissected out and assayed by immunofluorescence labeling for changes in LSR protein levels.

Results:

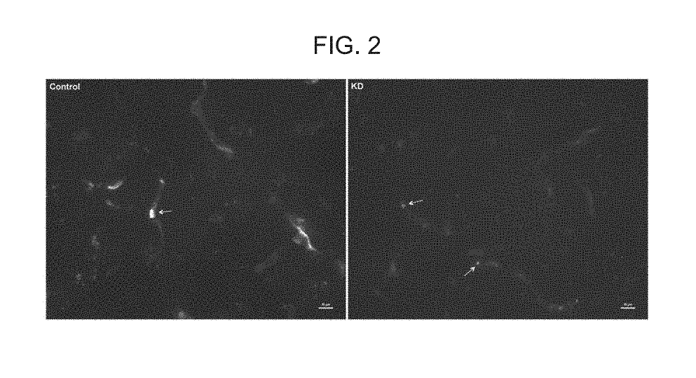

Representative immunofluorescence staining images labeled with anti-LSR antibody showing cerebral cortical sections from mice receiving siRNA#736+#2119 (KD) or scrambled siRNA (control) injections (see e.g., FIG. 2).

Note the tricellular tight junction in the cerebral capillary blood vessels (arrow). Note that KD samples showed reduced LSR protein labeling intensity.

### Example 4: In Vivo Knockdown of LSR Causes Increased Permeability of Fluorescein Across the BBB

The following example describes the in vivo knockdown of LSR causing increased permeability of fluorescein across the BBB into brain parenchyma.

Approach:

1.5 mg/kg BW-1 of mouse LSRm_siRNA_#736 and 1.5 mg/kg BW-1 of LSRm_siRNA_#2119 were mixed and injected with in vivo-jetPEI delivery reagent into mouse tail vein on day 0. On day 1, another dose of 1.5 mg/kg BW-1 of mouse LSRm_siRNA_#736 and 1.5 mg/kg BW-1 of LSRm_siRNA_#2119 was injected with in vivo-jetPEI delivery reagent into the same mouse tail vein to boost the effects. On day two, 50 mg/kg BW-1 of fluorescein (332 Da) was injected to the same mouse intraperitoneally. 1 hr after fluorescein injection, the mouse was perfused with 1×PBS to remove residue fluorescein in circulation. Then the cerebral cortex was dissected out, homogenized, and assayed for fluorescein levels that penetrated from the blood vessel across the BBB into the cerebral parenchyma tissues. The fluorescein levels were determined with a fluorescence reader at excitation wavelength of 488 nm and emission wavelength of 512 nm.

Results:

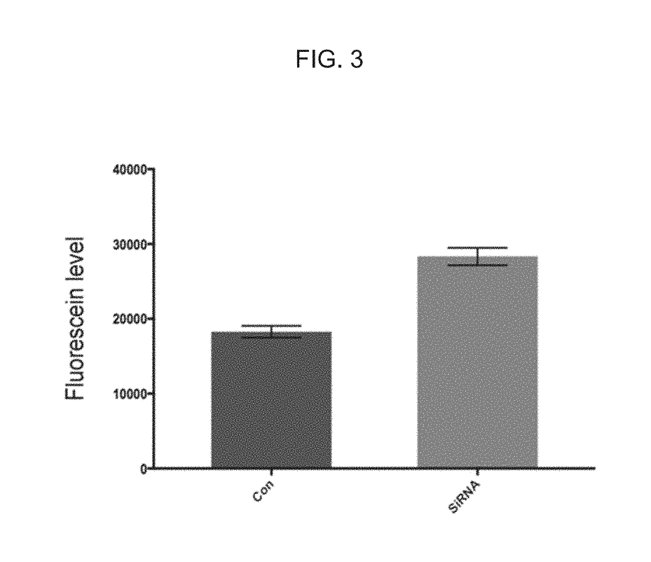

N=4 mice receiving LSR siRNA injection (KD) (siRNA #736+#2119); N=4 mice receiving scrambled siRNA injection (Control) (see e.g., FIG. 3). LSR siRNA injection significantly increased the fluorescein levels by 1.55-fold that have penetrated into the brain parenchyma from circulation across the BBB (p<0.05).

### Example 5: In Vivo Knockdown of LSR Causes Increased Permeability of Fluorescein-Temozolomide Conjugate Across the BBB

The following example describes the in vivo knockdown of LSR causing increased permeability of fluorescein-temozolomide conjugate across the BBB into brain parenchyma.

Approach:

1.5 mg/kg BW-1 of mouse LSRm_siRNA_#736 and 1.5 mg/kg BW-1 of LSRm_siRNA_#2119 were mixed and injected with in vivo-jetPEI delivery reagent into mouse tail vein on day 0. On day 1, another dose of 1.5 mg/kg BW-1 of mouse LSRm_siRNA_#736 and 1.5 mg/kg BW-1 of LSRm_siRNA_#2119 was injected with in vivo-jetPEI delivery reagent into the same mouse tail vein to boost the effects. On day two, 10 mg/kg BW-1 of Fluorescein-Temozolomide Conjugate (524 Da) was injected to the same mouse intraperitoneally. 1 hr after Fluorescein-Temozolomide Conjugate injection, the mouse was perfused with 1×PBS to remove residue Fluorescein-Temozolomide Conjugate in circulation. Then the cerebral cortex was dissected out, homogenized and assayed for Fluorescein-Temozolomide Conjugate levels that have penetrated from the blood vessel across the BBB into the cerebral parenchyma tissues. The Fluorescein-Temozolomide Conjugate levels were determined with a fluorescence reader at excitation wavelength of 488 nm and emission wavelength of 512 nm.

Results:

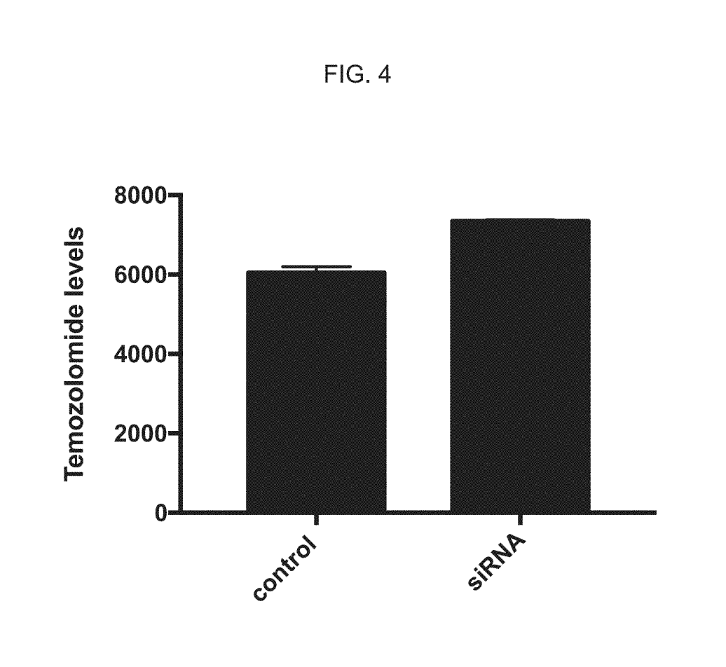

N=4 mice receiving LSR siRNA injection (KD) (siRNA #736+#2119); N=4 mice receiving scrambled siRNA injection (Control) (see e.g., FIG. 4). LSR siRNA injection significantly increased the Fluorescein-Temozolomide Conjugate levels by 1.22-fold that have penetrated into the brain parenchyma from circulation across the BBB (p<0.05).

### Example 6: In Vivo Knockdown of LSR Causes Increased Permeability of Fluorescein-Doxorubicin Conjugate Across the BBB

The following example describes the in vivo knockdown of LSR causing increased permeability of Fluorescein-doxorubicin Conjugate across the BBB into brain parenchyma.

Approach:

1.5 mg/kg BW-1 of mouse LSRm_siRNA_#736 and 1.5 mg/kg BW-1 of LSRm_siRNA_#2119 were mixed and injected with in vivo-jetPEI delivery reagent into mouse tail vein on day 0. On day 1, another dose of 1.5 mg/kg BW-1 of mouse LSRm_siRNA_#736 and 1.5 mg/kg BW-1 of LSRm_siRNA_#2119 was injected with in vivo-jetPEI delivery reagent into the same mouse tail vein to boost the effects. On day two, 10 mg/kg BW-1 of Fluorescein-doxorubicin Conjugate (933 Da) was injected to the same mouse intraperitoneally. 1 hr after Fluorescein-doxorubicin Conjugate injection, the mouse was perfused with 1×PBS to remove residue Fluorescein-doxorubicin Conjugate in circulation. Then the cerebral cortex was dissected out, homogenized and assayed for Fluorescein-doxorubicin Conjugate levels that have penetrated from the blood vessel across the BBB into the cerebral parenchyma tissues. The Fluorescein-doxorubicin Conjugate levels were determined with a fluorescence reader at excitation wavelength of 488 nm and emission wavelength of 512 nm.

Results:

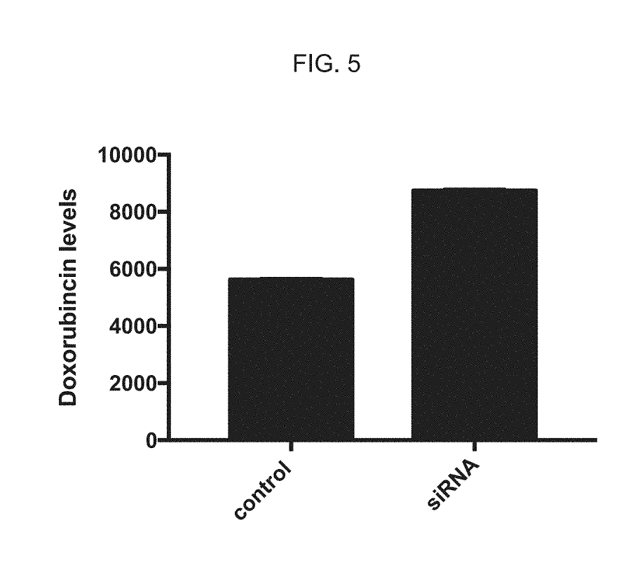

N=4 mice receiving LSR siRNA injection (KD) (siRNA #736+#2119); N=4 mice receiving scrambled siRNA injection (Control) (see e.g., FIG. 5). LSR siRNA injection significantly increased the Fluorescein-doxorubicin Conjugate levels by 1.55-fold that have penetrated into the brain parenchyma from circulation across the BBB (p<0.05).

In addition to demonstrating that the LSR knockdown technology is effective in a live adult mouse brain, it is further demonstrated that the LSR knockdown technology is also effective in vitro in cultured human blood brain barrier cells to increase its permeability (see e.g., Examples 7-10).

### Example 7: In Vitro Knockdown of LSR mRNA Levels in the Human Blood Vessel Cell Line

The following example describes the in vitro knockdown of LSR mRNA levels in the human blood vessel cell line—HCMEC/D3.

Approach:

30 pmol of human siRNA LSRh_siRNA_#216 and 30 pmol of human siRNA LSRh_siRNA_#822 were mixed and transfected into 1×10⁶ cells of the HCMEC/D3 line with the Lipofectamine 2000 reagent. The transfected cells were incubated at 37° C. for 48 hrs. Then, they were lysed and extracted for mRNA quantitative PCR analyses.

Results:

N=4 cell transfections receiving LSR siRNAs (KD) (siRNA #216+#822); N=4 cell transfections receiving scrambled siRNA (Control). LSR siRNA transfection significantly reduced the LSR expression levels in human blood vessel cell line HCMEC/D3 by 72% (p<0.05).

### Example 8: In Vivo Knockdown of LSR Causes Increased Permeability of Fluorescein Across the HCMEC/D3 Tight Junction

The following example describes the in vivo knockdown of LSR causing increased permeability of fluorescein across the HCMEC/D3 tight junction.

Approach:

30 pmol of human siRNA LSRh_siRNA_#216 and 30 pmol of human siRNA LSRh_siRNA_#822 were mixed and transfected into 1×10⁶ cells of the HCMEC/D3 line with the Lipofectamine 2000 reagent. The transfected cells were incubated at 37° C. for 48 hrs. Then, they were seeded onto Transwell to form tight junction barrier. 100 μM of fluorescein was loaded onto the apical side of the HCMEC/D3 cell monolayer. After 10 min of incubation at 37° C., the basolateral fluorescein levels were determined with a fluorescence reader at excitation wavelength of 488 nm and emission wavelength of 512 nm to reflect the fluorescein permeability across the tight junction barrier of the HCMEC/D3 cells.

Results:

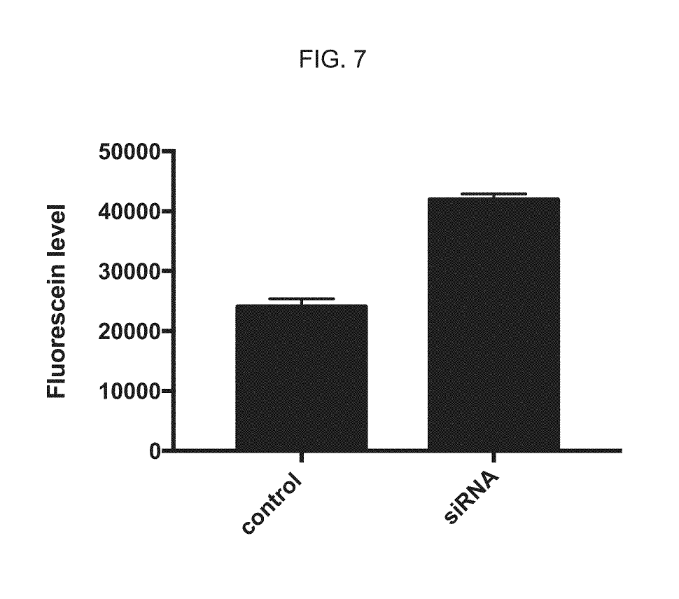

N=4 cell transfections receiving LSR siRNAs (KD) (siRNA #216+#822); N=4 cell transfections receiving scrambled siRNA (Control) (see e.g., FIG. 7). LSR siRNA transfection significantly increased the fluorescein levels by 1.74-fold that have crossed the tight junction of the HCMEC/D3 cells (p<0.05).

### Example 9: In Vivo Knockdown of LSR Causes Increased Permeability of Fluorescein-Temozolomide Conjugate Across the HCMEC/D3 Tight Junction

The following example describes the in vivo knockdown of LSR causing increased permeability of Fluorescein-Temozolomide Conjugate across the HCMEC/D3 tight junction.

Approach:

30 pmol of human siRNA LSRh_siRNA_#216 and 30 pmol of human siRNA LSRh_siRNA_#822 were mixed and transfected into 1×10⁶ cells of the HCMEC/D3 line with the Lipofectamine 2000 reagent. The transfected cells were incubated at 37° C. for 48 hrs. Then, they were seeded onto Transwell to form tight junction barrier. 50 μM of Fluorescein-Temozolomide Conjugate was loaded onto the apical side of the HCMEC/D3 cell monolayer. After 10 min of incubation at 37° C., the basolateral Fluorescein-Temozolomide Conjugate levels were determined with a fluorescence reader at excitation wavelength of 488 nm and emission wavelength of 512 nm to reflect the Fluorescein-Temozolomide Conjugate permeability across the tight junction barrier of the HCMEC/D3 cells.

Results:

N=4 cell transfections receiving LSR siRNAs (KD) (siRNA #216+#822); N=4 cell transfections receiving scrambled siRNA (Control). LSR siRNA transfection significantly increased the Fluorescein-Temozolomide Conjugate levels by 1.41-fold that have crossed the tight junction of the HCMEC/D3 cells (p<0.05).

### Example 10: In Vivo Knockdown of LSR Causes Increased Permeability of Fluorescein-Doxorubicin Conjugate Across the HCMEC/D3 Tight Junction

The following example describes the in vivo knockdown of LSR causing increased permeability of Fluorescein-doxorubicin Conjugate across the HCMEC/D3 tight junction.

Approach:

30 pmol of human siRNA LSRh_siRNA_#216 and 30 pmol of human siRNA LSRh_siRNA_#822 were mixed and transfected into 1×10⁶ cells of the HCMEC/D3 line with the Lipofectamine 2000 reagent. The transfected cells were incubated at 37° C. for 48 hrs. Then, they were seeded onto Transwell to form tight junction barrier. 50 μM of Fluorescein-doxorubicin Conjugate was loaded onto the apical side of the HCMEC/D3 cell monolayer. After 10 min of incubation at 37° C., the basolateral Fluorescein-doxorubicin Conjugate levels were determined with a fluorescence reader at excitation wavelength of 488 nm and emission wavelength of 512 nm to reflect the Fluorescein-doxorubicin Conjugate permeability across the tight junction barrier of the HCMEC/D3 cells.

Results:

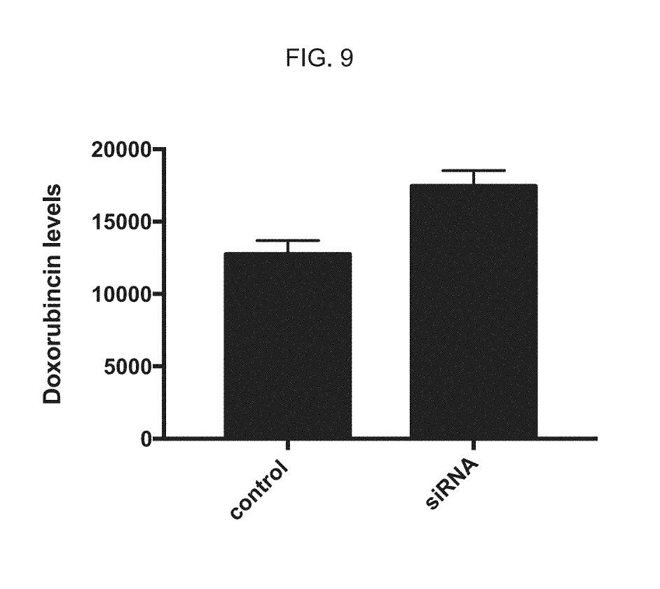

N=4 cell transfections receiving LSR siRNAs (KD) (siRNA #216+#822); N=4 cell transfections receiving scrambled siRNA (Control) (see e.g., FIG. 9). LSR siRNA transfection significantly increased the Fluorescein-doxorubicin Conjugate levels by 1.37-fold that have crossed the tight junction of the HCMEC/D3 cells (p<0.05).

### Example 11: Synthesis of FITC-Tagged Cancer Therapeutics, Permeability Testing, and Safety Testing

Synthesis of siRNA molecule is sufficient for 200 injections (1,000 mg of siRNA can be synthesized). For example, for the synthesis of fluorescence isoform of temozolomide and doxorubicin, 100 mg of FITC tagged temozolomide and doxorubicin can be made.

siRNA injection will be performed, (N=10 mice for each group, total of 40 mice) followed by assaying the brain abundance of FITC tagged temozolomide and doxorubicin to directly measure brain permeability of temozolomide and doxorubicin.

siRNA injection will be performed (N=10 mice for each group, total of 20 mice) followed by injecting a monodonal mouse anti-rabbit FITC tagged antibody to show that siRNA mediated BBB opening can facilitate antibody permeation through the barrier.

siRNA injection will be performed (N=10 mice for each group, total of 20 mice) followed by MRI analysis to determine specific brain regions most susceptible to siRNA induced BBB opening.

siRNA injection will be performed (N=10 mice for each group, total of 20 mice), followed by brain histology, electron microscopy analyses to determine if there is any morphologic and structural changes from the siRNA injection to brain tissue.

siRNA injection will be performed (N=10 mice for each group, total of 20 mice) followed by immunostaining for markers of apoptosis, activated microglial cells and damaged neurons to determine if there is any damage to neurons, glial cells, or microglial cells.

siRNA injection will be performed (N=10 mice for each group, total of 20 mice) through intrathecal route and assay for FITC tagged temozolomide permeability to determine if siRNA delivered to the cerebrospinal cord fluid may represent an alternative route for opening the BBB.

siRNA injection will be performed (N=10 mice for each group, total of 20 mice) followed by assaying liver function, renal function, and overall metabolism to determine if siRNA can cause any liver damage, renal damage, or affect overall animal metabolism.

Long-term siRNA injection will be performed (N=10 mice for each group, total of 20 mice) for 8 weeks at one injection per week to determine if prolonged and repeated siRNA injection may cause long-term damage to the brain, the liver and the kidney.

siRNA will be transfected to a human brain endothelial cell line and assayed for the measurement of the permeability of FITC tagged temozolomide and doxorubicin to show that the siRNA technology also works on human endothelial cells.

The siRNA technology will be further tested in primates to further demonstrate its safety in primate brains including behavior tests.

What is claimed is:

1. A method of increasing permeability of a tricellular junction in a blood brain barrier of a subject comprising administering an effective amount of a composition comprising a blood brain barrier opening agent, wherein the blood brain barrier opening agent comprises one or more compositions selected from the group consisting of:
(i) a synthetic RNA molecule, a RNA interference molecule, a siRNA, an antibody synthesized against a small molecule inhibitor of angulin;
(ii) an angulin inhibitor or an anti-angulin antibody;
(iii) a synthetic RNA molecule, a RNA interference molecule, or a siRNA synthesized against angulin; or
(iv) a monoclonal antibody, a polyclonal antibody, or an antigen binding fragment thereof comprising an antigen binding site that binds specifically to a LSR or ILDR polypeptide.

2. The method of claim 1, wherein the subject has a brain pathology.

3. The method of claim 1, wherein the effective amount of the blood brain barrier opening agent increases permeability of the tricellular junction.

4. The method of claim 1, wherein the blood brain barrier opening agent comprises one or more selected from the group consisting of:
(i) an anti-LSR (lipolysis stimulated lipoprotein receptor) antibody or an anti-ILDR (immunoglobulin-like domain containing receptor), an antigen binding fragment thereof, or a functional equivalent thereof, or a nucleic acid encoding the antibody thereof;
(ii) an RNAi molecule directed to LSR or ILDR, or a polynucleotide encoding the RNAi molecule;
(iii) an anti-LSR antibody that specifically binds to an epitope of the LSR;
(iv) an anti-ILDR antibody that specifically binds to an epitope of the ILDR;
(v) an anti-LSR antibody or anti-ILDR antibody is a monoclonal antibody; and
(vi) an anti-LSR antibody or anti-ILDR antibody is an antibody selected from the group consisting of: a monoclonal antibody, polyclonal antibody, chimeric antibody, humanized antibody, human antibody, multifunctional antibody, bispecific or oligospecific antibody, single chain antibody, scFV, diabody, sc(Fv)2 (single chain (Fv)2), and scFv-Fc.

5. The method of claim 1, wherein the blood brain barrier opening agent comprises:
an LSR siRNA, wherein the LSR siRNA reduces LSR cerebral expression; or
an ILDR siRNA, wherein the ILDR siRNA reduces ILDR cerebral expression.

6. The method of claim 1, wherein the synthetic RNA molecule is a functional siRNA duplex molecule comprising sense and anti-sense strands selected from one or more of the group consisting of:
SEQ ID NO: 1 or a sequence 90% identical thereto and SEQ ID NO: 2 or a sequence 90% identical thereto;
SEQ ID NO: 3 or a sequence 90% identical thereto and SEQ ID NO: 4 or a sequence 90% identical thereto;
SEQ ID NO: 5 or a sequence 90% identical thereto and SEQ ID NO: 6 or a sequence 90% identical thereto; and
SEQ ID NO: 7 or a sequence 90% identical thereto and SEQ ID NO: 8 or a sequence 90% identical thereto;
wherein,
the functional siRNA duplex molecule has siRNA activity against angulin.

7. The method of any one of claim 1, wherein the composition comprises
(i) the blood brain barrier opening agent coupled to a moiety selected from the group consisting of a drug, a radionuclide, an enzyme, a toxin, a therapeutic agent, and a chemotherapeutic agent; or
(ii) a pharmaceutically acceptable excipient, a preservative, a water solubility enhancing reagent, a label, or a tag.

8. The method of claim 1, further comprising:
administering a therapeutically effective amount of a therapeutic agent, wherein the therapeutic agent crosses the blood brain barrier in an increased amount compared to a control not receiving the blood brain barrier opening agent.

9. The method of claim 8, wherein the therapeutic agent comprises a cancer treatment or a chemotherapeutic agent for brain tumor treatment.

10. The method of claim 8, wherein the therapeutic agent comprises radiation therapy, antibody therapy, chemotherapy, photodynamic therapy, adoptive T cell therapy, T_{reg} depletion, surgery, or a combination therapy with conventional drugs.

11. The method of claim 8, wherein the therapeutic agent is selected from one or more of the group consisting of a cytotoxic drug, a tumor vaccine, bevacizumab, cetuximab, immunostimulatory antibodies, peptides, pepti-bodies, small molecules, a chemotherapeutic agent, interferons, interleukins, growth hormones, folic acid, vitamins, minerals, aromatase inhibitors, RNAi, histone deacetylase inhibitors, and proteasome inhibitors.

12. The method of claim 9, wherein the chemotherapeutic agent is selected from the group consisting of a cytotoxic agent and a cytostatic agent.

13. The method of claim 9, wherein the chemotherapeutic agent is selected from the group consisting of paclitaxel, cisplatin, vinorelbine, docetaxel, gemcitabine, temozolomide, irinotecan, 5FU, and carboplatin.

14. The method of claim 2, wherein the brain pathology is selected from the group consisting of a brain cancer, a brain tumor, a spinal cord cancer, a spinal cord tumor, a neurodegenerative disease, multiple sclerosis, stroke, or Alzheimer's disease.

15. The method of claim 2, wherein the brain pathology is a brain or spinal cord tumor selected from one or more of the group consisting of: Acoustic Neuroma; Astrocytoma; Atypical Teratoid Rhaboid Tumor (ATRT); Chordoma; Chondrosarcoma; Choroid Plexus; CNS Lymphoma; Craniopharyngioma; cysts; Ependymoma; Ganglioglioma; Germ Cell Tumor; Glioblastoma (GBM); Gliomas (e.g., Brain Stem Glioma, Ependymoma, Mixed Glioma, Optic Nerve Glioma, Subependymoma); Hemangioma; Lipoma; Lymphoma; Medulloblastoma; Meningioma; Metastatic Brain Tumors; Neurofibroma; Neuronal & Mixed Neuronal-Glial Tumors; Non-Hodgkin lymphoma; Oligoastrocytoma; Oligodendroglioma; Pineal Tumors; Pituitary Tumors; Primitive Neuroectodermal (PNET); Other Brain-Related Conditions; Schwannoma (neurilemmomas); Brain Stem Glioma; Craniopharyngioma; Ependymoma; Juvenile Pilocytic Astrocytoma (JPA); Medulloblastoma; Optic Nerve Glioma; Pineal Tumor; Primitive Neuroectodermal Tumors (PNET); and Rhabdoid Tumor.

16. The method of claim 1, wherein the administering of the composition comprising the blood brain barrier opening agent:
(i) results in slowed progression or amelioration of a brain pathology, a brain tumor, a brain cancer, a spinal cord tumor, a spinal cord cancer, or a neurological disease;
(ii) does not result in global tight junction breakdown; or
(iii) does not result in neuro-inflammation.

17. A method of producing a synthetic siRNA molecule against angulin comprising the steps of:
(i) providing a single stranded sense RNA molecule;
(ii) providing a single stranded anti-sense RNA molecule; and
(iii) combining the single stranded sense RNA molecule and the single stranded anti-sense RNA molecule, forming a functional siRNA duplex molecule;
wherein,
the functional siRNA duplex molecule has siRNA activity against angulin.

18. The method of claim 17, wherein the synthetic siRNA molecule is a functional siRNA duplex molecule comprising sense and anti-sense strands selected from one or more of the group consisting of:
SEQ ID NO: 1 or a sequence 90% identical thereto and SEQ ID NO: 2 or a sequence 90% identical thereto;
SEQ ID NO: 3 or a sequence 90% identical thereto and SEQ ID NO: 4 or a sequence 90% identical thereto;
SEQ ID NO: 5 or a sequence 90% identical thereto and SEQ ID NO: 6 or a sequence 90% identical thereto; and
SEQ ID NO: 7 or a sequence 90% identical thereto and SEQ ID NO: 8 or a sequence 90% identical thereto.

19. The method of claim 17, wherein the
(i) single stranded sense RNA molecule and single stranded antisense RNA molecule are chemically synthesized by automated solid phase oligonucleotide synthesizer; or
(ii) combining the single stranded sense RNA molecule and the single stranded anti-sense RNA molecule comprises approximately molar equivalents of a sense strand and an anti-sense strand.

20. The method of claim 19, further comprising combining the functional siRNA duplex molecule with a liposome reagent, forming an in vivo-grade siRNA molecule.


SEQUENCE LISTING (21 sequences)

SEQ ID NO: 1 - DNA, 21 bp, Artificial Sequence
  synthetic sequence
cttccagaatgcaacaggatt

SEQ ID NO: 2 - DNA, 21 bp, Artificial Sequence
  synthetic sequence
tcctgttgcattctggaagtt

SEQ ID NO: 3 - DNA, 21 bp, Artificial Sequence
  synthetic sequence
atgctgacctgacctttgatt

SEQ ID NO: 4 - DNA, 21 bp, Artificial Sequence
  synthetic sequence
tcaaaggtcaggtcagcattt

SEQ ID NO: 5 - DNA, 21 bp, Artificial sequence
  synthetic sequence
atgctgacctgaccttcgatt

SEQ ID NO: 6 - DNA, 21 bp, Artificial sequence
  synthetic sequence
tcgaaggtcaggtcagcattt

SEQ ID NO: 7 - DNA, 21 bp, Artificial sequence
  synthetic sequence
ttggaatattgatgaaacttt

SEQ ID NO: 8 - DNA, 21 bp, Artificial sequence
  synthetic sequence
agtttcatcaatattccaatt

SEQ ID NO: 9 - protein, 649 aa, Homo sapiens
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 10 - protein, 591 aa, homo sapiens
MQQDGLGVGTRNGSGKGRSVHPSWPWCAPRPLRYFGRDARARRAQTAAMALAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 11 - protein, 629 aa, homo sapiens
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 12 - protein, 630 aa, homo sapiens
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 13 - protein, 581 aa, homo sapiens
MQQDGLGVGTRNGSGKGRSVHPSWPWCAPRPLRYFGRDARARRAQTAAMALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLVYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 14 - protein, 493 aa, homo sapiens
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGMYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 15 - protein, 546 aa, homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEALARHRYMKQAQALGPQMMGKPLYWGADRSSQVSSYPMHPLLQRDLSLPSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPPFRSRCQERPRRPSPRESTQRHGRRRRHRSYSPPLPSGLSSWSSEEDKERQPQSWRAHRRGSHSPHWPEEKPPSYRSLDITPGKNSRKKGSVERRSEKDSSHSGRSVVI

SEQ ID NO: 16 - protein, 502 aa, homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEDLSLPSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPPFRSRCQERPRRPSPRESTQRHGRRRRHRSYSPPLPSGLSSWSSEEDKERQPQSWRAHRRGSHSPHWPEEKPPSYRSLDITPGKNSRKKGSVERRSEKDSSHSGRSVVI

SEQ ID NO: 17 - protein, 265 aa, homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEALARHRYMKQAQALGPQMMGKPLYWGADRSSQVSSYPMHPLLQRASRRCQ

SEQ ID NO: 18 - protein, 211 aa, homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCCCPEEALARHRYMKQAQALGPQMMGKPLYWGADRSSQVSSYPMHPLLQRDLSLPSSLPQMPMTQTTNQPPIANGVLEYLEKELRNLNLAQPLPPDLKGRFGHPCSMLSSLGSENQIEEF

SEQ ID NO: 19 - protein, 457 aa, homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNPLARHRYMKQAQALGPQMMGKPLYWGADRSSQVSSYPMHPLLQRDLSLPSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPPFRSRCQERPRRPSPRESTQRHGRRRRHRSYSPPLPSGLSSWSSEEDKERQPQSWRAHRRGSHSPHWPEEKPPSYRSLDITPGKNSRKKGSVERRSEKDSSHSGRSVVI

SEQ ID NO: 20 - protein, 514 aa, homo sapiens
MAGNIFCPFALFFLPMSRVGHLQHFLLLLALGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEDLSLPSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPPFRSRCQERPRRPSPRESTQRHGRRRRHRSYSPPLPSGLSSWSSEEDKERQPQSWRAHRRGSHSPHWPEEKPPSYRSLDITPGKNSRKKGSVERRSEKDSSHSGRSVVI

SEQ ID NO: 21 - protein, 639 aa, homo sapiens
MDRVLLRWISLFWLTAMVEGLQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVLLGVFLFFVLVGICWCQCCPHSCCCYVRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHEEDSNFRQSFHQMRSKQFPVSGDLESNPDYWSGVMGGSSGASRGPSAMEYNKEDRESFRHSQPRSKSEMLSRKNFATGVPAVSMDELAAFADSYGQRPRRADGNSHEARGGSRFERSESRAHSGFYQDDSLEEYYGQRSRSREPLTDADRGWAFSPARRRPAEDAHLPRLVSRTPGTAPKYDHSYLGSARERQARPEGASRGGSLETPSKRSAQLGPRSASYYAWSPPGTYKAGSSQDDQEDASDDALPPYSELELTRGPSYRGRDLPYHSNSEKKRKKEPAKKTNDFPTRMSLVV